(19) United States
(12) Patent Application Publication
(10) Pub. No.: US 2013/0324556 A1
(43) Pub. Date: Dec. 5, 2013

(54) Protease Activated Receptor 2 (PAR2) Antagonists

(71) Applicants: Joe William Boyd, London (GB); Paul Meo, Londo (GB); Michael Higginbottom, London (GB); Iain Simpson, London (GB); David Mark Mountford, London (GB); Edward Daniel Savory, London (GB)

(72) Inventors: Joe William Boyd, London (GB); Paul Meo, Londo (GB); Michael Higginbottom, London (GB); Iain Simpson, London (GB); David Mark Mountford, London (GB); Edward Daniel Savory, London (GB)

(21) Appl. No.: 13/981,960

(22) PCT Filed: Jan. 27, 2012
(86) PCT No.: PCT/GB12/50177

§ 371 Date: Aug. 22, 2013

(30) Foreign Application Priority Data
Jan. 28, 2011 (GB) 1101517.9

(51) Int. Cl. C07D 211/16; C07D 211/18; C07D 401/06; C07D 211/46; C07D 471/04; C07D 211/54; C07D 211/62; C07D 295/215; C07D 207/06; C07D 413/04; C07D 211/22; C07D 211/58

(52) U.S. Cl. CPC C07D 211/16; C07D 211/22; C07D 211/18; C07D 401/06; C07D 211/46; C07D 211/58; C07D 211/54; C07D 211/62; C07D 295/215; C07D 207/06; C07D 413/04; C07D 471/04
USPC 514/255.01; 546/226; 514/330; 546/194; 514/318; 546/221; 514/327; 546/223; 514/329; 546/225; 544/390; 548/538; 514/423; 546/189; 514/316; 546/198; 514/321; 546/199; 514/322; 546/113; 514/300

(57) ABSTRACT

A compound of formula (I) or a pharmaceutically acceptable salt, solvate, or hydrate thereof

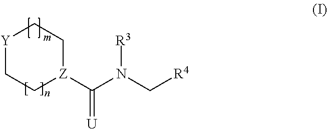

Wherein Y, Z, R³, U, R⁴, m and n are as defined in the claims.

### INTRODUCTION

This invention relates to compounds that are PAR2 receptor antagonists, to compositions containing them, to processes for their preparation, and to their use in medicine, in particular for the treatment of conditions which respond to antagonism of the PAR2 receptor, such as inflammation, intestinal inflammation, inflammatory skin diseases including psoriasis and itch, fibrosis, arthritis, pain, cancer and pancreatitis.

### BACKGROUND TO THE INVENTION

Protease activated receptors (PARs) are a family of seven transmembrane domain G-protein-coupled receptors that are activated by cleavage of their extracellular N-terminal domain by proteolytic enzymes. The newly exposed N-terminal sequence acts as a tethered ligand that binds to the extracellular face of the receptor and activates it. Four PARs have been described that are selectively cleaved by different enzymes; PAR1, PAR3 and PAR4 are cleaved by thrombin, PAR2 and PAR4 predominantly by trypsin and tryptase and PAR4 also cleaved by cathepsin G.

### PAR-2 in the GI Tract

The GI tract and pancreas are particularly exposed to a large array of proteases which can activate PAR2 receptors. Trypsin is released into the lumen of the pancreatic duct and the upper GI tract, for physiological digestive purposes. Other proteases abundant in the GI tract include those derived from enteric bacteria and those generated during disease processes. On mucosal surfaces, a balance between proteolytic activity and the presence of protease inhibitors such as pancreatic secretory trypsin inhibitor (PSTI) is constantly present.

PAR2 receptors are expressed throughout the GI tract specifically on mast cells, smooth muscle cells, myenteric neurons and endothelial cells, and on both the apical and basolateral sides of enterocytes (Kong et al., 1997). Since trypsin present in the GI lumen could activate PAR2 on apical surfaces, this receptor may provide a means by which the epithelium “senses” luminal processes.

In the gut, motility and secretion are regulated by neurons of the submucosal and myenteric plexi of the gastrointestinal tract. These neurones express PAR1, PAR2 and PAR4. PAR2 is expressed by secretomotor neurons in the submucosal plexus of the small intestine, where brief activation of PAR2 by agonists such as SLIGRL-NH₂ or trypsin results in a prolonged depolarisation that is often accompanied by increased excitability. Tryptase also induces a transient depolarization and a sustained increase in neuronal excitability (Linden et al., 2001). These observations indicate that PAR2 excites a proportion of myenteric neurons, which may contribute to dysmotility during intestinal inflammation.

A recent report concluded that activation of PAR2 in GI epithelial cells could trigger pro-inflammatory signalling including release of IL-8 via two independent pathways, MEK/ERK and PI3K/Akt. PAR2 would thus be confirmed to be a therapeutic target for treatment of inflammatory diseases of the GI tract (Tanaka et al., 2008). Histological studies in fibrotic intestine from patients with Crohn's disease indicated that (myo)fibroblasts are expanded in number and are the major cell types at sites of fibrosis in all layers of the intestinal wall (Pucilowska et al., 2000). Recent analysis of inflammatory tissues from patients with Crohn's Disease showed PAR2 over-expression in all cell types analyzed, including fibroblasts (Ketabchi et al., 2007). These results are in line with observations showing PAR2 over expression during fibrosis of lung and kidney (Cederqvist et al., 2005; Grandaliano et al., 2003). In these studies, PAR2 was identified as a potentially crucial receptor for the pathogenesis and sustainability of fibrosis. This hypothesis has recently been substantiated by a report linking normal activation of PAR2 by the protease Factor X resulting in tissue regeneration following injury, to the fibrotic response seen following repeated stimulation of this system as a consequence of chronic inflammatory bowel disease (IBD, Borensztajn et al., 2008).

In conclusion, PAR2 activation is important in the establishment, maintenance, and progression of intestinal inflammation and of fibrosis.

Itch Associated with Psoriasis and Atopic Dermatitis

Itch in human skin can be induced by both histamine and proteases. The ability of the PAR2 agonist SLIGRL-NH₂ to cause scratching behaviour in mice was not antagonised by antihistamine treatment (Shimada et al., 2006). The authors concluded that PAR2 was a histamine independent mediator of itch. Such an interpretation has been strengthened by the identification of the receptor on the terminals of sensory nerve fibres which transduce the itch sensation (Steinhoff et al., 2000). In non-GI tissues such as the skin, “tissue trypsins” are secreted which can activate PAR2 receptors, as can other proteases including tryptase, Factor X, Factor Vila and Tissue Factor (Bunnett, 2006). In patients with atopic dermatitis PAR-2 has also been strongly implicated as a major cause of itch (Steinhoff et al., 2003).

Psoriasis is a common skin condition which typically develops as patches (‘plaques’) of red, scaly skin. People with psoriasis have a faster turnover of skin cells associated with changes in the blood supply of the skin (redness) which causes local inflammation. Psoriasis is not due to an infection and is not infectious, nor is it cancerous.

Itch in psoriasis is a significant but often unrecognized problem in dermatology. A recent study found that itching was the most frequent complaint (64%) among patients hospitalised for psoriasis, (Sampogna et al., 2004) and several other studies confirm that itch is a principal symptom of psoriasis (Van de Kerkhof et al., 1998, 2000). Interestingly PAR2 receptors are highly expressed in the skin of psoriatic patients (Steinhoff et al., 1999), as are numerous tryptase-positive cells. These are found in the dermis and at the dermal-epidermal border in atopic dermatitis and psoriasis, and occasionally in the epidermis of psoriasis lesions. Tryptase released from such cells activates PAR2 in keratinocytes which may induce local inflammatory changes and thereby contribute to the pathophysiology of atopic dermatitis and psoriasis.

Furthermore it is hypothesised that other types of itch such as neuropathic itch are linked to an activation of PAR2 receptors by proteases (Binder et al., 2008).

For the forgoing reasons it is expected that a PAR2 antagonist will be effective in the treatment of inflammatory skin diseases including psoriasis and itch. In particular, it is expected that topical or systemic administration of a PAR2 antagonist would reduce the itch caused by local inflammation in psoriasis, and therefore would constitute a targeted treatment for this unchallenged symptom of psoriasis. It is also expected that a PAR2 antagonist will be effective in the treatment of arthritis due to inflammation in or around the joint.

### Pain

The transmission of pain and/or unpleasant sensation is also enhanced by activation of PAR2 receptors as application of activating peptide excites C fibres and sensitises them to heat (Ding-Pfennigdorf et al., 2004).

### Cancer

PAR-2 has been implicated in cellular proliferation, invasion and metastasis. There is increasing evidence that PAR2 is an important mediator of tumour progression, with trypsin levels being elevated in gastric, colon, ovarian and lung tumours (Ducroc et al., 2002). In addition PAR-2 is expressed in cancers of the lungs, liver, prostate, thyroid, breast, gastrium, colon, pancreas, gallbladder, melanoma and glioblastoma (see Jahan et al., 2007 and references therein).

Tissue factor (TF) is a primary component of the clotting cascade which with Factor Vila or Factor Xa can initiate clotting. Cancer patients are frequently in a pro-thrombotic state, apparently partly due to the release of TF containing microparticles (small membranous fragments perhaps released on apoptosis). TF is expressed at high levels in vessel wall fibroblasts but may also be expressed on endothelial and smooth muscle cells (Kasthuri et al., 2009). TF is also heavily implicated in cancer, its expression generally increasing with cancer stage (Kakkar et al., 1995; Kasthuri et al., 2009) and appears to be involved in metastasis (Belting et al., 2005). Indeed TF may play a role in forming the fibrinous clot around metastatic cells which serves to protect them from NK cells and to maintain them in the vasculature (Palumbo et al., 2005, 2007).

TF/Factor VIIa/Factor Xa complexes stimulate breast carcinoma cell migration and invasion through activation of PAR2 (Hjortoe et al., 2004; Morris et al., 2006). In addition in other cancers including colon and gastric carcinomas, activated PAR2 stimulates EGFR activity and thus cellular proliferation (Caruso et al., 2006); Darmoul et al., 2004). Indeed in ovarian cancer increase in PAR-2 was seen with progression of the cancer irrespective of the histopathological classification of the tumour type, and high cancer cell PAR-2 expression was associated with a significantly worse prognosis (Jahan et al., 2007). Similarly patients with lymph node metastases of uterine cancers with high levels of PAR-2 had significantly worse prognosis than those with lower levels (Jahan et al., 2008). PAR-2 has also been implicated in tumour angiogenesis in cancers of the breast, colon, gastrium, pancreas, lungs, prostate, melanoma and glioblastoma (see Jahan et al., 2007).

### Pancreatitis

Pancreatitis is an inflammatory condition understood to be the result of undesirable trypsin activity within the pancreas. The biological effects of trypsin in the pancreas have been shown to act through PAR2, which is strongly expressed on the luminal surface of acinar and ductal cells (Ceppa et al., 2011; Laukkarinen et al., 2008). Antagonism of the effects of trypsin at PAR2, within the pancreas, is expected to be an effective treatment for pancreatitis.

### Inflammation

PAR2 receptor activation has been shown to be important in inflammatory disorders. Based on in vivo studies in models of inflammatory disorders (Kelso et al, JPET, 2006, 316, 1017-1024, Sevigny, PNAS, 2011, 108, 20, 8491-8496 and Cenac et al, JDR, 2010, 89, 10, 1123-1128) it is expected that antagonism of the PAR2 receptor will be effective in the treatment of inflammatory disorders.

For the above reasons, the PAR2 receptor is regarded as a target for intervention in the treatment of the conditions referred to above. There are few antagonists of PAR-2 available which are suitable for therapeutic treatment. Accordingly a small molecule antagonist is desirable for therapy.

### BRIEF DESCRIPTION OF THE INVENTION

This invention makes available a class of compounds which are antagonists of the PAR2 receptor, and their use in indications which respond to the antagonism of the PAR2 receptor such as those mentioned above.

### DETAILED DESCRIPTION OF THE INVENTION

According to the present invention there is provided a compound of formula (I) or a pharmaceutically acceptable salt thereof:

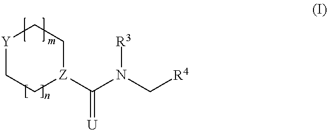

Y is —N(R^{1A})— or —C(R^{1B})(R²)—; and

R^{IA} is —X—R⁵ and R^{1B} is -Q-R⁵;

X is independently selected from a direct bond, —C(O)—, —(CHR⁶)ₚ—, —N(R⁶)— or, in either orientation, —(CH₂CHR⁶)—;

Q is independently selected from a direct bond, —O—, S, —N(R⁶)—, —C(O)—, C(H)(OH)—, —(CHR⁶)ₚ— or, in either orientation, —(CH₂CHR⁶)—;

p is 1 or 2;

U═O or S

R⁵ is a monocyclic aromatic or non-aromatic carbocyclic or heterocyclic ring having 5 or 6 ring atoms, optionally fused to a second aromatic or non-aromatic monocyclic carbocyclic or heterocyclic ring to form a 5-5, 5-6, 6-5, or 6-6 bicyclic ring system, which monocyclic ring or bicyclic ring system is optionally substituted with one more substituents independently selected from halogen, hydroxy, cyano, nitro, CF₃, C₁₋₄-alkyl, C₁₋₄-alkoxy and —NR^{7A}R^{7B}, wherein

R^{7A}, R^{7B} are each independently selected from hydrogen and C₁₋₄-alkyl, wherein any alkyl residue is optionally substituted with one or more substituents independently selected from fluorine, hydroxyl and C₁₋₄-alkoxy,

or

R^{7A} and R^{7B}, together with the nitrogen atom to which they are bound, form a 4- to 7-membered saturated heterocyclic ring, optionally substituted with one or more substituents independently selected from fluorine, hydroxyl, C₁₋₄-alkyl, fluoro-C₁₋₄-alkyl and C₁₋₄-alkoxy;

R² is H,

Z is N or CH, and the ring comprising Z and Y is optionally substituted,

n=0, 1, or 2, and m=0 or 1, provided that m=0 when n=2, and provided that neither m nor n=0 when Z and Y are each N, and

R³ and R⁶ are each independently selected from H, C₁₋₄ alkyl, or cyclopropyl each of which C₁₋₄ alkyl, or cyclopropyl being optionally substituted with one or more substituents independently selected from fluoro and C₁₋₄ alkoxy;

R⁴ is

(i) a 6-5 bicyclic ring system selected from

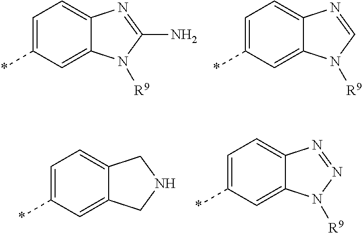

optionally substituted on either ring, and wherein the bond marked * is connected to the CH₂, or

(ii) the 5-6 bicyclic ring system

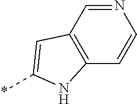

optionally substituted on either ring, and wherein the bond marked * is connected to the CH₂, or

(iii) a radical of formula —(W)ᵥ(CH₂)ₜB
wherein W is an optionally substituted phenyl or pyridyl ring, v is 0 or 1, and t is 0 or 3 provided that when v=0, t=3, and when v=1, t=0; and
B is selected from:

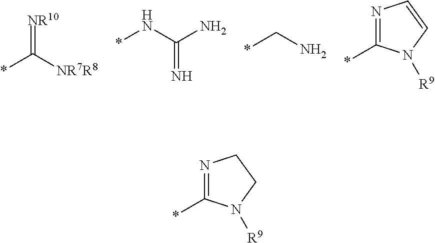

wherein R⁷, R⁸, R⁹ and R¹⁰ are independently selected from H, C₁₋₄ alkyl, or cyclopropyl, each of which C₁₋₄ alkyl, or cyclopropyl being optionally substituted with one or more substituents independently selected from fluoro and C₁₋₄ alkoxy; or
R⁷ and R⁸ together with the nitrogen atom to which they are attached form a 3-5 membered heterocyclic ring selected from aziridine, azetidine, and pyrrolidine each of which being optionally substituted with one or more substituents independently selected from fluoro and C₁₋₄ alkoxy.

Compounds of formula (I) above may be prepared in the form of salts, especially pharmaceutically acceptable salts, N-oxides, hydrates, solvates and polymorphic forms thereof. Any claim to a compound herein, or reference herein to “compounds of the invention”, “compounds with which the invention is concerned”, “compounds of formula (I)” and the like, includes salts, N-oxides, hydrates, solvates and polymorphs of such compounds;

Although the above definition potentially includes molecules of high molecular weight, it is preferable, in line with general principles of medicinal chemistry practice, that the compounds with which this invention is concerned should have molecular weights of no more than 600.

The compounds of the invention are antagonists of the PAR2 receptor. Therefore, in another broad aspect the invention provides the use of a compound of the invention in the treatment of, or in the preparation of a composition for treatment of, diseases or conditions responsive to the reduction of PAR2 mediated activity.

Examples of diseases or conditions which are responsive to the reduction of PAR2 mediated activity include inflammation such as intestinal inflammation and inflammatory skin diseases including psoriasis and itch, fibrosis, arthritis, pain and cancers including cancers of the breast, colon, gastrium, pancreas, lungs, prostate, melanoma and glioblastoma, and pancreatitis.

The compounds with which the invention is concerned may be used for the reduction of PAR2 mediated activity, ex vivo or in vivo.

In one aspect of the invention, the compounds of the invention may be used in the preparation of a composition for the treatment of conditions including inflammation such as intestinal inflammation and inflammatory skin diseases including psoriasis and itch, fibrosis, arthritis, pain and cancers including cancers of the breast, colon, gastrium, pancreas, lungs, prostate, melanoma and glioblastoma, and pancreatitis

In another aspect, the invention provides a method for the treatment of the foregoing disease types, which comprises administering to a subject suffering such disease an effective amount of a compound of the invention.

In another aspect of the invention there is provided a pharmaceutical composition comprising a compound as claimed in any of the preceding claims, together with one or more pharmaceutically acceptable carriers and/or excipients.

The compounds of the invention may be administered in a variety of dosage forms. Thus, they can be administered orally, for example as tablets, capsules, troches, lozenges, aqueous or oily suspensions, dispersible powders or granules.

The compounds can be administered in a sublingual formulation, for example a buccal formulation. The compounds of the invention may also be administered parenterally, whether subcutaneously, intravenously, intramuscularly, intrasternally, transdermally, by inhalation, intranasally, or by infusion techniques. The compounds may also be administered as suppositories. The compounds may also be administered topically. Thus, the compounds of the invention are administered orally, or by inhalation, topically, or intranasally. In a preferred embodiment, the compounds of the invention are administered orally and more preferably, the compounds of the invention are administered as a tablet or capsule. In the latter connection, administration of the compounds in a hard gelatine capsule form, or in one of the many sustained release formulations known in the art will often be preferred. In an alternative preferred embodiment the compounds of the invention are administered as a topical treatment.

The present invention further provides a pharmaceutical composition containing a compound of the invention or a pharmaceutically acceptable salt thereof, as defined above, and a pharmaceutically acceptable carrier.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Since the compounds of the invention are preferably administered orally, the present invention further provides a pharmaceutical composition containing a compound of the invention or a pharmaceutically acceptable salt thereof, as defined above, and a pharmaceutically acceptable carrier in the form of a capsule or tablet.

Alternatively, the compounds of the invention are preferable administered topically. The compounds may be formulated in any form suitable for topical administration including semi-solid, spray, medicated powders, solution, and medicated adhesive systems. Once formulated, the compounds of the invention may be administered as external topicals that are spread, sprayed, or otherwise dispersed on to cutaneous tissues to cover the affected area. Topical drug delivery is especially effective in the fields of psoriasis, itch, and pain management.

Solutions for injection or infusion may contain as carrier, for example, sterile water or preferably they may be in the form of sterile, aqueous, isotonic saline solutions.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the art. However, it is expected that a typical dose will be in the range from about 0.001 to 50 mg per kg of body weight.

### Terminology

The following definitions shall apply throughout the specification and the appended claims, unless otherwise stated or indicated.

Where elements present in the compounds of the invention exist as different isotopes, for example carbon (C¹³ and C¹⁴) nitrogen (N¹⁴ and N¹⁵) and hydrogen (H¹ and H² i.e. deuterium), such compounds form part of the invention irrespective of the isotopic form of the element present in the compound. In particular, where a compound of the invention has a hydrogen atom in any position, that hydrogen may be replaced by deuterium. It is known in the art that deuterium substitution can increase the metabolic stability of biologically active molecules.

The term “C_{a-b}-alkyl” wherein a and b are integers denotes a straight or branched alkyl group having from a to b carbon atoms. For example “C₁₋₄-alkyl” includes methyl, ethyl, n-propyl, isopropyl, n-butyl, iso-butyl, sec-butyl and tert-butyl and “C₁₋₆-alkyl” includes the foregoing and straight- and branched-chain pentyl and hexyl.

The term “fluoro-C_{a-b}-alkyl” wherein a and b are integers denotes a straight or branched C_{a-b}-alkyl group substituted by one or more fluorine atoms. For example fluoro-C₁₋₄-alkyl includes fluoromethyl, trifluoromethyl, 2-fluoroethyl and 2,2,2-trifluoroethyl.

The term “C_{a-b}-alkoxy” wherein a and b are integers refers to a straight or branched C_{a-b}-alkyl group which is attached to the remainder of the molecule through an oxygen atom. For example C₁₋₄-alkoxy includes methoxy, ethoxy, n-propoxy, iso-propoxy, n-butoxy, iso-butoxy, sec-butoxy and tert-butoxy.

The term “fluoro-C_{a-b}-alkoxy” wherein a and b are integers denotes a fluoro-C_{a-b}-alkyl group which is attached to the remainder of the molecule through an oxygen atom. For example “fluoro-C₁₋₄-alkoxy” groups include trifluoromethoxy and 2,2,2-trifluoroethoxy.

The term “C_{a-b}-alkoxy-C_{c-d}-alkyl” wherein a, b, c and d are integers denotes a straight or branched alkoxy group having from a to b carbon atoms connected to a straight or branched alkyl group having from c to d carbon atoms. For example “C₁₋₄-alkoxy-C₁₋₄-alkyl” includes methoxymethyl, methoxyethyl, ethoxyethyl, iso-propoxyethyl, n-butoxyethyl and tert-butoxyethyl.

The term fluoro-C_{a-b}-alkoxy-C_{c-d}-alkyl wherein a, b, c and d are integers denotes a C_{a-b}-alkoxy-C_{c-d}-alkyl group substituted by one or more fluorine atoms. For example “fluoro-C₁₋₄-alkoxy-C₁₋₄-alkyl” includes trifluoromethoxymethyl and trifluoromethoxyethyl.

The term “C_{a-b}-cycloalkyl” wherein a and b are integers denotes a saturated monocyclic hydrocarbon ring having from a to b carbon atoms. For examples “C₃₋₅-cycloalkyl” includes cyclopropyl, cyclobutyl and cyclopentyl.

The term “C_{a-b}-cycloalkyl-C_{c-d}-alkyl” wherein a, b, c and d are integers denotes a saturated monocyclic hydrocarbon ring having from a to b carbon atoms connected to a straight or branched alkyl group having from c to d carbon atoms. For example “C₃₋₅-cycloalkyl-C₁₋₄-alkyl” includes cyclopropylmethyl and cyclobutylmethyl.

As used herein the term “carbocyclic” refers to a mono-, bi- or tricyclic radical having up to 16 ring atoms, all of which are carbon, and includes aryl and cycloalkyl.

Unless otherwise particularised, the term “heterocyclyl” or “heterocyclic ring” denotes a saturated, monocyclic ring having from 4 to 7 ring atoms with at least one heteroatom such as O, N, or S, and the remaining ring atoms are carbon. Examples of heterocyclic rings include piperidinyl, tetrahydropyranyl, tetrahydrofuranyl, oxetanyl, azetidinyl, pyrrolidinyl, morpholinyl, thiomorpholinyl, dioxanyl, piperazinyl and homopiperazinyl. When present, the sulfur atom may be in an oxidized form (i.e., S═O or O═S═O). Exemplary heterocyclic groups containing sulfur in oxidized form are 1,1-dioxido-thiomorpholinyl and 1,1-dioxido-isothiazolidinyl.

Unless otherwise particularised the term “heterocyclyl-C_{a-b}-alkyl” wherein a and b are integers denotes a heterocyclic ring as defined above that is directly attached to a straight or branched C_{a-b}-alkyl group via a carbon or nitrogen atom of said ring. For example “heterocyclyl-C₁₋₄-alkyl” groups include piperidin-1-ylmethyl, piperidin-4-ylmethyl and morpholin-4-ylmethyl.

Unless otherwise particularised the term “heteroaryl” denotes a monocyclic or fused bicyclic heteroaromatic ring system comprising 5 to 10 ring atoms in which one or more of the ring atoms are other than carbon, such as nitrogen, sulphur or oxygen. Only one ring need be aromatic and said heteroaryl moiety can be linked to the remainder of the molecule via a carbon or nitrogen atom in any ring. Examples of heteroaryl groups include furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, oxadiazolyl, thiadiazolyl, benzofuranyl, 2,3-dihydrobenzofuranyl, 1,3-benzodioxolyl, 1,4-benzodioxinyl, benzothiazolyl, benzimidazolyl, azabenzimidazole, benzotriazolyl and chromanyl.

Unless otherwise particularised the term “C_{a-b}-aryl” wherein a and b are integers denotes a monocyclic or fused bicyclic hydrocarbon ring system comprising a to b ring atoms and wherein at least one ring is an aromatic ring. For example “C₆₋₁₀-aryl” groups include phenyl, indenyl, 2,3-dihydroindenyl (indanyl), 1-naphthyl, 2-naphthyl or 1,2,3,4-tetrahydronaphthyl.

Unless otherwise particularised the term “C_{a-b}-aryl-C_{c-d}-alkyl” wherein a, b, c and d are integers refers to a C_{a-b}-aryl group that is directly linked to a straight or branched C_{c-d-}alkyl group. For example “C₆₋₁₀-aryl-C₁₋₄-alkyl” groups include phenylmethyl (i.e., benzyl) and phenylethyl.

Unless otherwise particularised the term “heteroaryl-C_{a-b}-alkyl” wherein a and b are integers denotes a heteroaryl ring as defined above that is directly linked to a straight or branched C_{a-b-}alkyl group via a carbon or nitrogen atom of said ring. For examples “heteroaryl-C₁₋₄-alkyl” groups include 2-(pyridin-2-yl)-ethyl and 1,2,4-oxadiazol-5-ylmethyl.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto (C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

As used herein the term “salt” includes base addition, acid addition and ammonium salts. As briefly mentioned above compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds of the invention which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, trifluoroacetic, tartaric, succinic, fumaric, maleic, malic, salicylic, citric, methanesulphonic, p-toluenesulphonic, benzoic, benzenesulfonic, glutamic, lactic, and mandelic acids and the like. Those compounds (I) which have a basic nitrogen can also form quaternary ammonium salts with a pharmaceutically acceptable counter-ion such as chloride, bromide, acetate, formate, p-toluenesulfonate, succinate, hemi-succinate, naphthalene-bis sulfonate, methanesulfonate, trifluoroacetate, xinafoate, and the like. For a review on salts, see Handbook of Pharmaceutical Salts: Properties, Selection, and Use by Stahl and Wermuth (Wiley-VCH, Weinheim, Germany, 2002).

It is expected that compounds of the invention may be prepared in the form of hydrates, and solvates. Any reference herein, including the claims herein, to “compounds with which the invention is concerned” or “compounds of the invention” or “the present compounds”, and the like, includes reference to salts, hydrates, and solvates of such compounds. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Individual compounds of the invention may exist in an amorphous form and/or several polymorphic forms and may be obtained in different crystal habits. Any reference herein, including the claims herein, to “compounds with which the invention is concerned” or “compounds of the invention” or “the present compounds”, and the like, includes reference to the compounds irrespective of amorphous or polymorphic form.

Some compounds of the invention, having a nitrogen atom in an aromatic ring, may form N-oxides, and the invention includes compounds of the invention in their N-oxide form.

In the compounds of the invention, in any compatible combination, and bearing in mind that the compounds preferably have a molecular weight of less than 600.

### The Group X and Q

As defined above, X is independently selected from a direct bond, —C(O)—, —(CHR⁶)ₚ— with p being 1 or 2, for example —(CH₂)ₚ—, —(CHCH₃)ₚ—, —(CHCH₂CH₃)ₚ—, —(CHCH₂CH₂CH₃)ₚ, —(CHCH(CH₃)₂)ₚ—, —(CHCH(CH₂)₃)ₚ—, —(CHC(CH₃)₃)ₚ—, —N(R⁶) such as —NH, —N(CH₃), —N(CH₂CH₃), —N(CH₂CH₂CH₃), —N(CH₂C(CH₃)₂), or —NCH(CH₂)₃ or, in either orientation, —(CH₂CHR⁶)—, for example —(CH₂CHCH₃)—;

Q is independently selected from a direct bond, —O—, —S—, —N(R⁶)—, —C(O)—, C(H)(OH)—, —(CHR⁶)ₚ— or, in either orientation, —(CH₂CHR⁶)—, wherein, for example, each of —N(R⁶)—, —(CHR⁶)ₚ—, and —(CH₂CHR⁶)— are defined for group X above.

In an embodiment of the invention X is independently selected from —C(O)—, —(CHR⁶)ₚ—, —N(R⁶)— or, in either orientation, —(CH₂CHR⁶)—.

In an alternative embodiment of the invention Q is independently selected from —O—, —S—, —N(R⁶)—, —C(O)—, C(H)(OH)—, —(CHR⁶)ₚ— or, in either orientation, —(CH₂CHR⁶)—.

The group U is either O (an oxygen atom), or S (a sulfur atom). In a preferred embodiment U is O.

The groups R³, R^{6,} R⁷, R⁸, R⁹, and R¹⁰

As defined above, R³, R⁶, R⁷, R⁸, R⁹, and R¹⁰ are each independently selected from H, C₁₋₄ alkyl such as methyl, ethyl, n-propyl, iso-propyl, n-butyl, iso-butyl, and sec-butyl, or cyclopropyl, and each of which C₁₋₄ alkyl or cyclopropyl being optionally substituted with one or more substituents independently selected from fluoro, and C₁₋₄ alkoxy such as methoxy, ethoxy, n-propoxy, iso-propoxy, n-butoxy, iso-butoxy, sec-butoxy and tert-butoxy. In an embodiment of the invention R¹⁰ is hydrogen. In an alternative embodiment R¹⁰ is C₁₋₄ alkyl

### The Group R⁵

As defined above R⁵ is a monocyclic aromatic or non-aromatic carbocyclic or heterocyclic ring having 5 or 6 ring atoms such as phenyl, pyridyl, piperidine, pyrrole, imidazole, imidazoline, imidazolone, optionally fused to a second aromatic or non-aromatic monocyclic carbocyclic or heterocyclic ring such as phenyl or pyridyl to form a 5-5, 5-6, 6-5, or 6-6 bicyclic ring system, such as which monocyclic ring or bicyclic ring system is optionally substituted with one more substituents independently selected from halogen such as fluoro or chloro, hydroxy, cyano, nitro, CF₃, C₁₋₄-alkyl such as methyl, or ethyl, C₁₋₄-alkoxy and NR^{7A}R^{7B}, wherein

R^{7A}, R^{7B} are each independently selected from hydrogen and C₁₋₄-alkyl, wherein any alkyl residue is optionally substituted with one or more substituents independently selected from fluorine, hydroxyl and C₁₋₄-alkoxy,

or

R^{7A} and R^{7B}, together with the nitrogen atom to which they are bound, form a 4- to 7-membered saturated heterocyclic ring, optionally substituted with one or more substituents independently selected from fluorine, hydroxyl, C₁₋₄-alkyl, fluoro-C₁₋₄-alkyl and C₁₋₄-alkoxy;

### The Group B

As defined above, B is selected from:

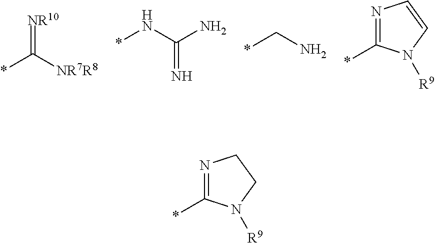

wherein R⁷, R⁸, R⁹ and R¹⁰ are independently selected from H, or C₁₋₄ alkyl such as methyl, ethyl, n-propyl, iso-propyl, n-butyl, iso-butyl, and sec-butyl, or cyclopropyl, and each of which C₁₋₄ alkyl or cyclopropyl being optionally substituted with one or more substituents independently selected from fluoro, chloro and bromo, and C₁₋₄ alkoxy such as methoxy, ethoxy, n-propoxy, iso-propoxy, n-butoxy, iso-butoxy, sec-butoxy and tert-butoxy;

or R⁷ and R⁸ together with the nitrogen atom to which they are attached form a 3-5 membered heterocyclic ring selected from aziridine, azetidine, and pyrrolidine each of which being optionally substituted with one or more substituents independently selected from fluoro, chloro and bromo, and C₁-C₄ alkoxy such as methoxy, ethoxy, n-propoxy, iso-propoxy, n-butoxy, iso-butoxy, sec-butoxy and tert-butoxy.

In a presently preferred embodiment of the invention, R⁷ and R⁸ are independently selected from H, C₁₋₄ alkyl, or cyclopropyl, each of which C₁₋₄ alkyl, or cyclopropyl being optionally substituted with one or more substituents independently selected from fluoro, and C₁-C₄ alkoxy.

In another presently preferred embodiment the ring comprising Z and Y is selected from:

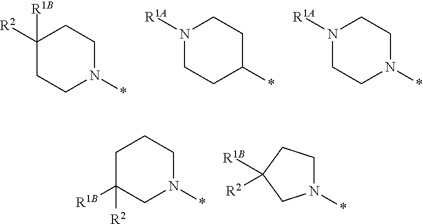

in which the bond marked * connects to the carbon of the carbonyl group. Yet more preferably the ring comprising Z and Y is optionally substituted with one more substituents independently selected from fluoro, C₁₋₄-alkyl such as methyl, ethyl, n-propyl, iso-propyl, n-butyl, iso-butyl, and sec-butyl, C₁₋₄-alkoxy such as methoxy, ethoxy, n-propoxy, iso-propoxy, n-butoxy, iso-butoxy, sec-butoxy and tert-butoxy, fluoro-C₁₋₄-alkyl such as fluoromethyl, trifluoromethyl, 2-fluoroethyl and 2,2,2-trifluoroethyl, and fluoro-C₁₋₄-alkoxy such as trifluoromethoxy and 2,2,2-trifluoroethoxy.

In another preferred embodiment the radical —(W)ᵥ(CH₂)ₜB is selected from:

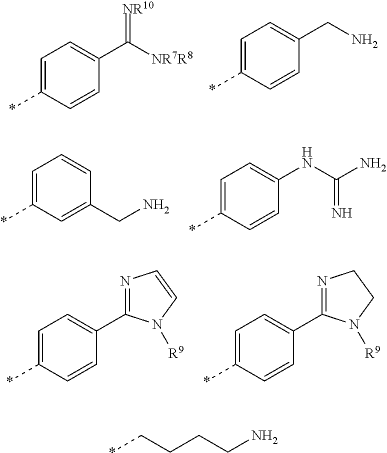

any of which being optionally substituted, and wherein the bond marked * is connected to the CH₂ of the rest of the molecule, and R⁷, R⁸, R⁹ and R¹⁰ are as previously defined. Yet more preferably, the phenyl ring is substituted with one or more fluoro substituents.

In a presently preferred embodiment the radical —(W)ᵥ(CH₂)ₜB has v=1, and W is an optionally substituted phenyl or pyridyl ring. Preferably W is an optionally substituted phenyl ring. In a yet further preferred embodiment the group B and the CH₂ of the rest of the molecule are connected to the W ring in a para arrangement.

In a yet further preferred embodiment R⁴ is selected from:

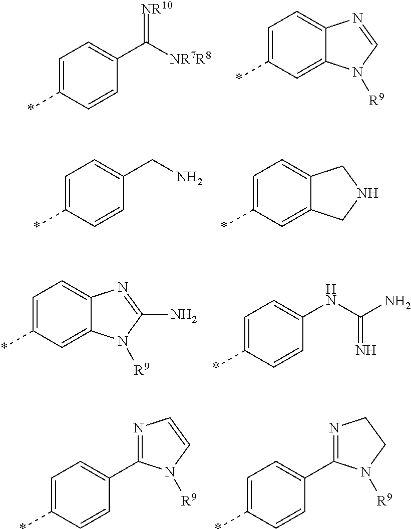

each of which being optionally substituted, and wherein the bond marked * is connected to the CH₂ of the rest of the molecule, and R⁷, R⁸, R⁹ and R¹⁰ are as previously defined. Yet more preferably, the phenyl ring is substituted with one or more fluoro substituents, preferably one or two or three fluoro substituents.

In a particularly preferred embodiment R³ is H.

In another particularly preferred embodiment Z is N═.

In an alternative particularly preferred embodiment R⁶ is H or methyl.

In a yet further preferred embodiment R⁹ is H or methyl.

In another presently preferred embodiment R⁵ is selected from:

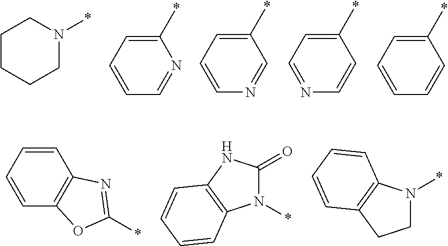

wherein the bond marked * connects R⁵ to the rest of the molecule, each of which being optionally substituted with one more substituents independently selected from halogen, hydroxy, cyano, nitro, CF₃, C₁₋₄-alkyl such as methyl, ethyl, C₁₋₄-alkoxy such as methoxy, ethoxy, and NR^{7A}R^{7B}, wherein R^{7A}, R^{7B} are each independently selected from hydrogen and C₁₋C₄-alkyl, wherein any alkyl residue is optionally substituted with one or more substituents independently selected from fluorine, hydroxyl and C₁₋₄-alkoxy,
or

R^{7A} and R^{7B}, together with the nitrogen atom to which they are bound, form a 4- to 7-membered saturated heterocyclic ring, optionally substituted with one or more substituents independently selected from fluorine, hydroxyl, C₁₋C₄-alkyl, fluoro-C₁₋C₄-alkyl and C₁₋C₄-alkoxy.

Specific examples of compounds according to the invention include:
(4-{[(4-Benzylpiperidin-1-yl)carbonylamino]methyl}phenyl)methanaminium chloride;
2,2,2-Trifluoroacetic acid; N-{[4-(aminomethyl)phenyl]methyl}-4-[(4-methoxyphenyl)methyl]piperidine-1-carboxamide;
2,2,2-Trifluoroacetic acid; N-{[4-(aminomethyl)phenyl]methyl}-4-[(3-fluorophenyl)methyl]piperidine-1-carboxamide;
N-{[4-(Aminomethyl)phenyl]methyl}-4-[(4-fluorophenyl)methyl]piperidine-1-carboxamide hydrochloride;
2,2,2-Trifluoroacetic acid; N-{[4-(aminomethyl)phenyl]methyl}-4-[(4-chlorophenyl)methyl]piperidine-1-carboxamide;
2,2,2-Trifluoroacetic acid; N-{[4-(aminomethyl)phenyl]methyl}-4-[(4-methylphenyl)methyl]piperidine-1-carboxamide
N-{[4-(Aminomethyl)phenyl]methyl}-4-(pyridin-2-ylmethyl)piperidine-1-carboxamide;
N-{[4-(Aminomethyl)phenyl]methyl}-4-(pyridin-4-ylmethyl)piperidine-1-carboxamide;
N-{[4-(Aminomethyl)phenyl]methyl}-4-(4-fluorophenoxy)piperidine-1-carboxamide;
N-{[4-(Aminomethyl)phenyl]methyl}-4-(phenylsulfanyl)piperidine-1-carboxamide hydrochloride;
N-{[4-(Aminomethyl)phenyl]methyl}-4-[(2-chlorophenyl)amino]piperidine-1-carboxamide dihydrochloride;
2,2,2-Trifluoroacetic acid; N-{[4-(aminomethyl)phenyl]methyl}-4-[(4-fluorophenyl)carbonyl]piperidine-1-carboxamide;
N-{[4-(Aminomethyl)phenyl]methyl}-4-[(4-fluorophenyl)(hydroxy)methyl]piperidine-1-carboxamide;
N-{[4-(Aminomethyl)phenyl]methyl}-1-[(3-fluorophenyl)methyl]piperidine-4-carboxamide;
N-{[4-(Aminomethyl)phenyl]methyl}-4-benzylpiperazine-1-carboxamide;
N-{[4-(Aminomethyl)phenyl]methyl}-4-[(2-chlorophenyl)methyl]piperazine-1-carboxamide dihydrochloride;
N-{[4-(Aminomethyl)phenyl]methyl}-4-(1,3-benzoxazol-2-yl)piperidine-1-carboxamide;
N-{[4-(Aminomethyl)-3-fluorophenyl]methyl}-4-benzylpiperidine-1-carboxamide;
4-Benzyl-N-[(4-carbamimidoylphenyl)methyl]piperidine-1-carboxamide;
2,2,2-Trifluoroacetic acid; 4-benzyl-N-{[4-(N,N-dimethylcarbamimidoyl)phenyl]methyl}piperidine-1-carboxamide;
N-[(4-Carbamimidoylphenyl)methyl]-4-(pyridin-4-ylmethyl)piperidine-1-carboxamide;
N-(1H-1,3-Benzodiazol-6-ylmethyl)-4-benzylpiperidine-1-carboxamide;
2,2,2-Trifluoroacetic acid; N-(1H-1,3-benzodiazol-5-ylmethyl)-3-phenylpyrrolidine-1-carboxamide;
2,2,2-Trifluoroacetic acid; N-(1H-1,3-benzodiazol-5-ylmethyl)-3-benzylpyrrolidine-1-carboxamide;
N-[(2-Amino-1H-1,3-benzodiazol-6-yl)methyl]-4-benzylpiperidine-1-carboxamide;
2,2,2-Trifluoroacetic acid; 4-benzyl-N-[(4-carbamimidamidophenyl)methyl]piperidine-1-carboxamide;
4-Benzyl-N-(2,3-dihydro-1H-isoindol-5-ylmethyl)piperidine-1-carboxamide

### Synthesis

The compounds of formula (I) above may be prepared by, or in analogy with, conventional methods. The preparation of intermediates and compounds according to the examples of the present invention may in particular be illustrated by, but not limited to, the following Schemes.

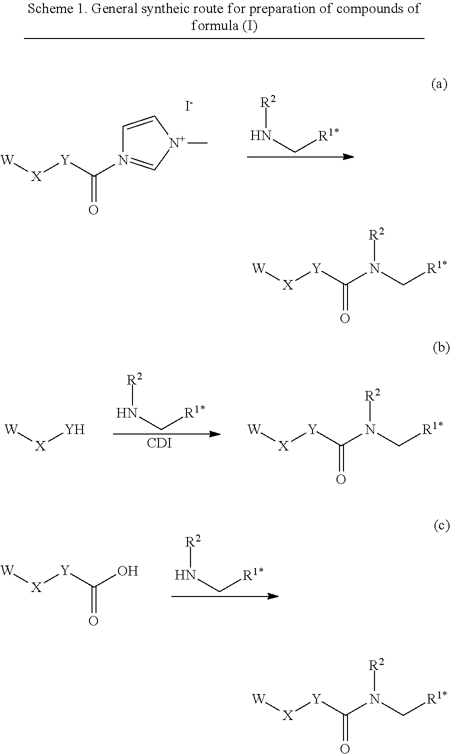

wherein, R^{1*} is either R¹ or a functional group that can be readily converted in to R¹.

The compounds of Formula (I) above may be prepared by the condensation of the appropriate primary amine, NH(R²)CH₂R^{1*} with (a) activated ureas, (b) amines or (c) carboxylic acids using standard procedures. All of these alternatives are exemplified in the experimental section below.

The following abbreviations have been used:

| | |
|---|---|
| AcOH | Acetic acid |
| aq | aqueous |
| Boc | tert-Butyloxycarbonyl |
| BSA | Bovine serum albumin |
| CDI | Carbonyl diimidazole |
| DBU | 1,8-Diazabicycloundec-7-ene |
| DCM | Dichloromethane |
| DIAD | Diisopropyl azodicarboxylate |
| DIPEA | N,N-Diisopropylethylamine |
| DMF | N,N-Dimethylformamide |
| DMSO | Dimethyl sulfoxide |
| DPPA | Diphenylphosphoryl azide |
| EDTA | Ethylenediaminetetraacetic acid |
| ES⁺ | Electrospray |
| ESI⁺ | Electrospray ionization |
| Et | Ethyl |
| Et₃N | Triethylamine |
| Et₂O | Diethyl ether |
| EtOAc | Ethyl acetate |
| EtOH | Ethanol |
| Ex | Example |
| HBSS | Hank's Buffered Salt Solution |
| HBTU | 2-(1H-Benzotriazole-1-yl)-1,1,3,3-tetramethyluronium |
| | hexafluorophosphate |
| HEPES | 4-(2-Hydroxyethyl)-1-piperazineethanesulfonic acid |
| HPLC | High Performance Liquid Chromatography |
| HRMS | High Resolution Mass Spectrometry |
| Int | Intermediate |
| LCMS | Liquid Chromatography Mass Spectrometry |
| M | Molar |
| MeCN | Acetonitrile |
| MeOH | Methanol |
| [MH]⁺ | Protonated molecular ion |
| MSD-TOF | Mass Selective Detector-Time of Flight |
| PAR | Protease activated receptor |
| PBS | Phosphate buffered saline |
| Rf | Retention time |
| sat | saturated |
| tBu | Tert-butyl |
| TFA | Trifluoroacetic acid |
| THF | Tetrahydrofuran |
| TLC | Thin Layer Chromatography |

### EXAMPLES AND INTERMEDIATE COMPOUNDS

### Experimental Methods

All reagents were commercial grade and were used as received without further purification, unless otherwise specified. Reagent grade solvents were used in all cases. Analytical LCMS was performed on either an Agilent 1100 system equipped with a Phenomenex Synergi, RP-Hydro, 150×4.6 mm, 4 μm column (MeCN in water (+0.085% TFA), 200-300 nm, 30° C.) or Agilent 1100 system equipped with a Phenomenex Gemini, C18, 100×4.6 mm, 4 μm column (MeCN in water (10 mM ammonium bicarbonate), 200-300 nm, 40° C.) (Marked # in text below). High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Analytical HPLC was performed on either an Agilent 1100 system using a Phenomenex Synergi, RP-Hydro, 150×4.6 mm, 4 m column with a flow rate of 1.5 mL per min at 30° C. (200-300 nm) and a gradient of either 5-100% MeCN (+0.085% TFA) in water (+0.1% TFA) over 7 min, 50-100% MeCN (+0.085% TFA) in water (+0.1% TFA) over 7 min (marked ** in text below), 5-50% MeCN (+0.085% TFA) in water (+0.1% TFA) over 7 min (marked *** in text below) or 5-95% MeCN (+0.085% TFA) in water (+0.1% TFA) over 20 min (marked * in text below). Flash chromatography was performed on either a CombiFlash Companion system equipped with RediSep silica columns or a Flash Master Personal system equipped with Strata SI-1 silica gigatubes or in a glass column under gravity. Reverse Phase HPLC was performed on a Gilson system (Gilson 322 pump with Gilson 321 equilibration pump and Gilson 215 autosampler) equipped with Phenomenex Synergi Hydro RP 150×10 mm, or YMC ODS-A 100/150×20 mm columns, or on an XTerra Prep MS C18 5 um 19×50 mm system. Microwave irradiations were carried out using a Biotage microwave. Reactions were performed at room temperature unless otherwise stated. The compounds were automatically named using ACD 6.0. All compounds were dried in a vacuum oven overnight. Where yields are not included, the intermediates were used crude. Reactions were monitored by TLC, LCMS or HPLC.

### Intermediate 1

### Tert-butyl 4-[(2-chlorophenyl)amino]piperidine-1-carboxylate

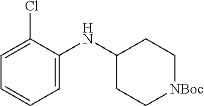 [SMILES: CC(C)(C)OC(=O)N1CCC(Nc2ccccc2Cl)CC1]

1-Chloro-2-iodobenzene (1.31 g, 5.49 mmol) was dissolved in dioxane (10 mL) and ^{t}BuONa (672 mg, 6.99 mmol), Pd₂(dba)₃ (183 mg, 0.20 mmol), Xantphos (326 mg, 0.40 mmol) and tert-butyl 4-aminopiperidine-1-carboxylate (1.00 g, 4.99 mmol) were added. The reaction mixture was heated at 100° C. for 3 d. The solvents were removed in vacuo and the residue was dissolved in DCM and filtered. The residue was purified by column chromatography to give the title compound (826 mg, 53%) as a light yellow oil. LCMS: ES⁺ 255.5 [MH]⁺-^{t}Bu.

### Intermediate 2

### 2,2,2-Trifluoroacetic acid; N-(2-chlorophenyl)piperidin-4-amine

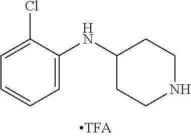 [SMILES: Clc1ccccc1NC1CCNCC1]

Intermediate 1 (826 mg, 2.66 mmol) was dissolved in DCM (20 mL) and TFA (3 mL) was added. The reaction mixture was stirred at room temperature for 18 h. The solvents were removed in vacuo to give the title compound (500 mg, 58%) as a light brown gum. LCMS: purity 100%, ES⁺ 211.5 [MH]⁺.

### Intermediate 3

### tert-Butyl N-({4-[(methylamino)methyl]phenyl}methyl)carbamate

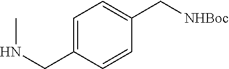 [SMILES: CNCc1ccc(CNC(=O)OC(C)(C)C)cc1]

Methanesulfonyl chloride (171 μL, 2.21 mmol) was dissolved in DCM (15 mL) and cooled to 0° C. A solution of tert-butyl N-{[4-(hydroxymethyl)phenyl]methyl}carbamate (500 mg, 2.11 mmol) and Et₃N (316 μL, 2.21 mmol) in DCM (5 mL) was added, the reaction mixture was stirred for 2 h and added drop-wise to a solution of 2.0 M methylamine in THF (50 mL). The reaction mixture was stirred for 18 h, poured into 1 M aq Na₂CO₃ (100 mL) and extracted with DCM (3×100 mL). The combined organic fractions were dried (MgSO₄) and concentrated in vacuo to give the crude title compound as a yellow oil (607 mg) which was used without further purification. LCMS: purity 80%, ES⁺ 251.6 [MH]⁺.

### Intermediate 4

### 4-Benzyl-1-(1H-imidazol-1-ylcarbonyl)piperidine

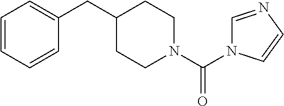 [SMILES: O=C(N1CCC(Cc2ccccc2)CC1)n1ccnc1]

CDI (23.1 g, 143 mmol) was dissolved in DCM (300 mL) and cooled to 0° C. 4-Benzylpiperidine (25.1 mL, 143 mmol) was added and the reaction mixture was stirred for 2 h. DCM (200 mL) was added and the reaction mixture was washed with 10% aq citric acid (2×250 mL), sat aq NaHCO₃ (250 mL) and water (250 mL), dried (MgSO₄) and the solvents were removed in vacuo to give the title compound as a yellow oil (36.0 g, 94%) which was used without further purification. LCMS: purity 100%, ES⁺ 270.1 [MH]⁺.

### Intermediate 5

### 4-Phenyl-1-(1H-imidazol-1-ylcarbonyl)piperidine

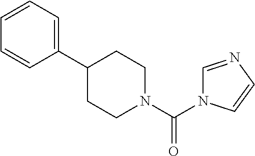 [SMILES: O=C(N1CCC(c2ccccc2)CC1)n1ccnc1]

The title compound (645 mg, 81%) was prepared similarly to Intermediate 4, using 4-phenyl-piperidine instead of 4-benzylpiperidine, as an off-white solid.

LCMS: ES⁺ 256.6 [MH]⁺.

### Intermediate 6

### 1-[(4-Benzylpiperidin-1-yl)carbonyl]-3-methyl-1H-imidazol-3-ium iodide

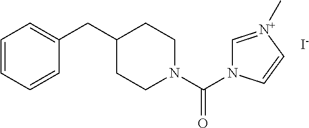 [SMILES: C[n+]1ccn(C(=O)N2CCC(Cc3ccccc3)CC2)c1 ; [I-]]

Intermediate 4 (36.0 g, 133 mmol) was dissolved in Et₂O (200 mL) and MeI (20.7 mL, 333 mmol) was added. The reaction mixture was stirred for 18 h and the precipitate was collected by filtration and washed with Et₂O (2×100 mL) to give the title compound (26.6 g, 49%) as a pale yellow solid which was used without further purification. LCMS: purity 87%, ES⁺ 284.2 [MH]⁺.

### Intermediate 7

### 1-[(4-Phenylpiperidin-1-yl)carbonyl]-3-methyl-1H-imidazol-3-ium iodide

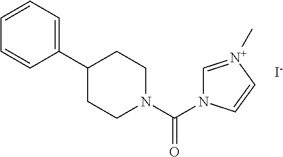 [SMILES: C[n+]1ccn(C(=O)N2CCC(c3ccccc3)CC2)c1 ; [I-]]

The title compound (121 mg, crude) was prepared similarly to Intermediate 6, using Intermediate 5 instead of Intermediate 4, as a white solid. LCMS: ES⁺ 270.6 [MH]⁺.

### Intermediate 8

### 2,2,2-Trifluoroacetic acid; 4-(aminomethyl)-2-fluorobenzonitrile

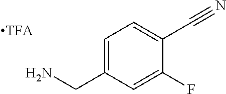 [SMILES: N#Cc1ccc(CN)cc1F]

4-Bromomethyl-2-fluoro-benzonitrile (500 mg, 2.34 mmol) and di-tert-butyl iminodicarboxylate (507 mg, 2.34 mmol) were dissolved in THF and cooled to 0° C. NaH (93.6 mg, 60% dispersion in mineral oil, 2.34 mmol) was added portion-wise and the reaction mixture stirred at 0° C. for 2 h. The reaction mixture was quenched with sat aq NH₄Cl (10 mL) and extracted with EtOAc (2×20 mL). The combined organic fractions were washed with brine (30 mL) and concentrated in vacuo. The residue was dissolved in Et₂O (10 mL), filtered and concentrated in vacuo. The residue was dissolved in DCM (10 mL) and TFA (2.5 mL) was added. The reaction mixture was stirred for 1 h and the solvents were removed in vacuo to give the crude title compound as a yellow oil which was used without further purification.

LCMS: ES⁺ 151.4 [MH]⁺.

### Intermediate 9

### 2-{[4-(1H-Imidazol-2-yl)phenyl]methyl}-2,3-dihydro-1H-isoindole-1,3-dione

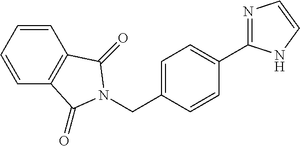 [SMILES: O=C1c2ccccc2C(=O)N1Cc1ccc(-c2ncc[nH]2)cc1]

[4-(1H-Imidazol-2-yl)phenyl]methanol (250 mg, 1.44 mmol), phthalamide (253 mg, 1.72 mmol) and triphenylphosphine (420 mg, 1.58 mmol) were dissolved in THF (8 mL) and stirred for 10 min. DIAD (0.31 mL, 1.58 mmol.) was added drop-wise over 2 min and the reaction mixture was stirred for 2 h. The solvents were removed in vacuo and the residue was purified by column chromatography. The residue was dissolved in EtOAc (15 mL), sonicated and the precipitate was collected by filtration to give the title compound (174 mg, 40%) as a white solid.

LCMS: ES⁺ 304.5 [MH]⁺.

### Intermediate 10

### [4-(1H-Imidazol-2-yl)phenyl]methanamine

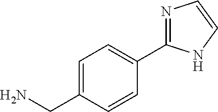 [SMILES: NCc1ccc(-c2ncc[nH]2)cc1]

Intermediate 9 (170 mg, 0.57 mmol) was dissolved in EtOH (15 mL), hydrazine hydrate (0.65 mL, 55% aq solution, 11.4 mmol) was added and the reaction mixture was stirred for 3 h. The solvents were removed in vacuo and the product dissolved in EtOAc (20 mL), sonicated and filtered. The solvents were removed in vacuo to give the title compound as a yellow oil which was used without further purification. LCMS: ES⁺ 174.4 [MH]⁺.

### Intermediate 11

### 1H-1,3-Benzodiazol-6-ylmethanol

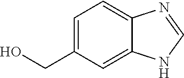 [SMILES: OCc1ccc2nc[nH]c2c1]

1H-Benzimidazole-5-carboxylic acid (5.00 g, 30.8 mmol) was dissolved in THF (100 mL) and cooled to 0° C. Lithium aluminium hydride (50.0 mL, 2.4 M in THF, 120 mmol) was added drop-wise and the reaction mixture was stirred at room temperature for 4 d. The reaction mixture was cooled to 0° C., quenched cautiously with 1 M aq NaOH, filtered and the solvents were removed in vacuo. The residue was purified by column chromatography to give the title compound (872 mg, 19%) as a yellow oil. LCMS: ES⁺ 149.4 [MH]⁺.

### Intermediate 12

### 1H-1,3-Benzodiazol-6-ylmethanamine

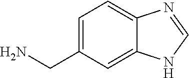 [SMILES: NCc1ccc2nc[nH]c2c1]

Intermediate 11 (604 mg, 4.08 mmol) was dissolved in THF (10 mL), DPPA (1.35 g, 4.89 mmol) and DBU (745 mg, 4.89 mmol) were added and the reaction stirred at room temperature for 4 h. The reaction mixture was concentrated in vacuo and diluted with EtOAc (50 mL). The organic layer was washed with sat aq NaCl solution (2×50 mL), dried (MgSO₄) and concentrated in vacuo. The residue was dissolved in THF (10 mL), LiAlH₄ solution (1.7 mL, 2.4 M in THF, 4.08 mmol) was added and the reaction mixture stirred at room temperature for 4 d. The reaction mixture was cooled to 0° C., quenched with MeOH, diluted with water and the solvents were removed in vacuo. The residue was purified by column chromatography to give the title compound (403 mg, 67%) as a yellow gum. LCMS: ES⁺ 148.4 [MH]⁺.

### Intermediate 13

### 2-Amino-1H-1,3-benzodiazole-6-carboxamide

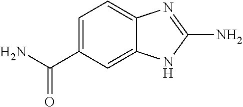 [SMILES: NC(=O)c1ccc2nc(N)[nH]c2c1]

3,4-Diaminobenzoic acid (200 mg, 1.31 mmol) was dissolved in MeOH (5 mL) and cyanogen bromide (170 mg, 1.64 mmol) was added. The reaction mixture was stirred for 1 h, the solvents were removed in vacuo and the residue and K₂CO₃ (180 mg, 1.31 mmol) were dissolved in DMF (2.5 mL). The reaction mixture was stirred for 10 min and HBTU (740 mg, 1.97 mmol), DIPEA (0.91 mL, 5.24 mmol) and NH₄Cl (140 mg, 2.62 mmol) were added. The reaction mixture was stirred for 16 h, diluted with EtOAc, filtered and the solvents were removed in vacuo. The residue was dissolved in DCM (20 mL), sonicated, and the resulting solid was collected by filtration to give the crude title compound (0.05 g, 22%) which was used without further purification. LCMS: ES⁺ 177.2 [MH]⁺.

### Intermediate 14

### 6-(Aminomethyl)-1H-1,3-benzodiazol-2-amine

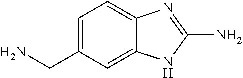 [SMILES: NCc1ccc2nc(N)[nH]c2c1]

Intermediate 13 (0.25 g, 1.42 mmol) was dissolved in THF (20 mL) and LiAlH₄ (1.24 mL, 2.4 M in THF, 2.98 mmol) was added. The reaction mixture was heated at reflux for 16 h, quenched with 1 M aq NaOH, filtered through celite and the solvents were removed in vacuo. The residue was dissolved in DCM (15 mL) and sonicated and the resulting precipitate was collected by filtration and purified by column chromatography to give the title compound (30.0 mg, 13%) as a yellow/brown gum. LCMS: ES⁺ 163.2 [MH]⁺.

### Intermediate 15

### 1H-1,2,3-Benzotriazole-6-carboxamide

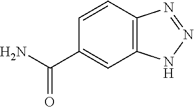 [SMILES: NC(=O)c1ccc2nn[nH]c2c1]

1H-1,2,3-Benzotriazole-5-carboxylic acid (500 mg, 3.10 mmol) and HBTU (1.87 g, 4.94 mmol) were dissolved in DMF (7 mL) and stirred for 15 min. NH₄Cl (330 mg, 6.20 mmol) and DIPEA (2.16 mL, 12.4 mmol) were added and the reaction mixture was stirred for 2 h. The solvents were removed in vacuo and the residue was tritiurated from MeOH/DCM (1:4) to give the title compound (406 mg, 81%) as a light brown solid. LCMS: ES⁺ 163.1 [MH]⁺.

### Intermediate 16

### 1H-1,2,3-Benzotriazol-6-ylmethanamine

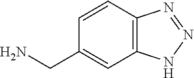 [SMILES: NCc1ccc2nn[nH]c2c1]

Intermediate 15 (100 mg, 0.62 mmol) was dissolved in THF (5 mL), LiAlH₄ (0.55 mL, 1.30 mmol) was added and the reaction mixture was heated at reflux for 16 h. The reaction mixture was quenched with 1M aq NaOH, filtered through celite and purified by column chromatography to give the crude title compound (51 mg) as an off-white solid which was used without further purification. LCMS: ES⁺ 149.1 [MH]⁺.

### Intermediate 17

### General Procedure A

### Tert-butyl N-[(4-{[(4-benzylpiperidin-1-yl)carbonylamino]methyl}phenyl)methyl]carbamate

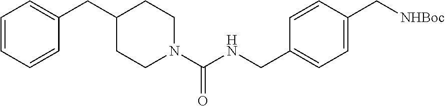 [SMILES: CC(C)(C)OC(=O)NCc1ccc(CNC(=O)N2CCC(Cc3ccccc3)CC2)cc1]

CDI (210 mg, 1.30 mmol) was dissolved in DCM (5 mL) and cooled to 0° C. A solution of tert-butyl N-{[4-(aminomethyl)phenyl]methyl}carbamate (300 mg, 1.27 mmol) and DIPEA (0.23 mL, 1.33 mmol) in DCM (5 mL) was added and the reaction mixture was warmed to room temperature over 18 h. A solution of 4-benzyl piperidine (0.22 mL, 1.25 mmol) and DIPEA (0.23 mL, 1.33 mmol) in DCM (5 mL) was added and the reaction mixture was stirred for 65 h, diluted with DCM (10 mL), washed with 1 M aq Na₂CO₃ (2×25 mL), dried (MgSO₄) and concentrated in vacuo. The residue was purified by column chromatography to give the title compound (365 mg, 66%) as an off-white solid. LCMS: purity 96.3%, ES⁺ 438.8 [MH]⁺.

### Intermediates 18-46

Intermediates 18-46 were prepared similarly to General Procedures A; see Table 1 below.

TABLE 1
Preparation of intermediate Boc protected amines.

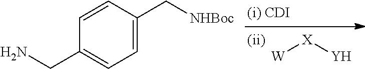

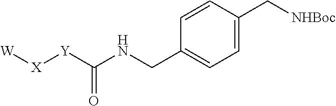

| Int | W—X—YH | Intermediate Name |
|---|---|---|
| 18 | 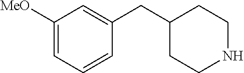 | Tert-butyl N-({4-[({4-[(3-methoxyphenyl)methyl]piperidin- 1-yl}carbonylamino)methyl]phenyl}methyl)carbamate |
| 19 | 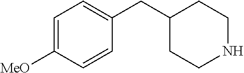 | Tert-butyl N-({4-[({4-[(4-methoxyphenyl)methyl]piperidin- 1-yl}carbonylamino)methyl]phenyl}methyl)carbamate |
| 20 | 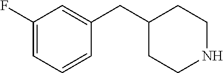 | Tert-butyl N-({4-[({4-[(3-fluorophenyl)methyl]piperidin-1- yl}carbonylamino)methyl]phenyl}methyl)carbamate |
| 21 | 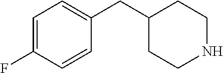 | Tert-butyl N-({4-[({4-[(4-fluorophenyl)methyl]piperidin-1- yl}carbonylamino)methyl]phenyl}methyl)carbamate |
| 22 | 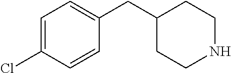 | Tert-butyl N-({4-[({4-[(4-chlorophenyl)methyl]piperidin-1- yl}carbonylamino)methyl]phenyl}methyl)carbamate |
| 23 | 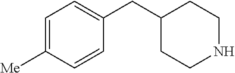 | Tert-butyl N-({4-[({4-[(4-methylphenyl)methyl]piperidin-1- yl}carbonylamino)methyl]phenyl}methyl)carbamate |
| 24 | 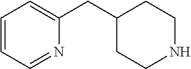 | Tert-butyl N-{[4-({[4-(pyridin-2-ylmethyl)piperidin-1- yl]carbonylamino}methyl)phenyl]methyl}carbamate |
| 25 | 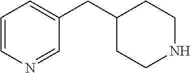 | Tert-butyl N-{[4-({[4-(pyridin-3-ylmethyl)piperidin-1- yl]carbonylamino}methyl)phenyl]methyl}carbamate |
| 26 | 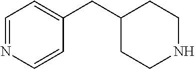 | Tert-butyl N-{[4-({[4-(pyridin-4-ylmethyl)piperidin-1- yl]carbonylamino}methyl)phenyl]methyl}carbamate |
| 27 | 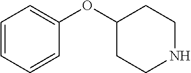 | Tert-butyl N-[(4-{[(4-phenoxypiperidin-1- yl)carbonylamino]methyl}phenyl)methyl]carbamate |
| 28 | 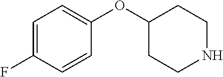 | Tert-butyl N-{[4-({[4-(4-fluorophenoxy)piperidin-1- yl]carbonylamino}methyl)phenyl]methyl}carbamate |
| 29 | 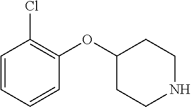 | Tert-butyl N-{[4-({[4-(2-chlorophenoxy)piperidin-1- yl]carbonylamino}methyl)phenyl]methyl}carbamate |
| 30 | 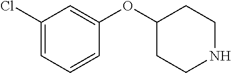 | Tert-butyl N-{[4-({[4-(3-chlorophenoxy)piperidin-1- yl]carbonylamino}methyl)phenyl]methyl}carbamate |
| 31 | 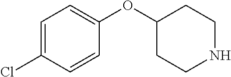 | Tert-butyl N-{[4-({[4-(4-chlorophenoxy)piperidin-1- yl]carbonylamino}methyl)phenyl]methyl}carbamate |
| 32 | 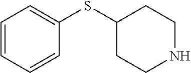 | Tert-butyl N-{[4-({[4-(phenylsulfanyl)piperidin-1- yl]carbonylamino}methyl)phenyl]methyl}carbamate |
| 33 | 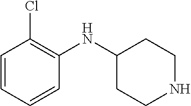 | Tert-butyl N-({4-[({4-[(2-chlorophenyl)amino]piperidin-1- yl}carbonylamino)methyl]phenyl}methyl)carbamate |
| 34 | 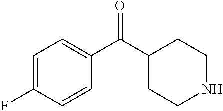 | Tert-butyl N-({4-[({4-[(4-chlorophenyl)carbonyl]piperidin-1- yl}carbonylamino)methyl]phenyl}methyl)carbamate |
| 35 | 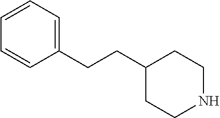 | Tert-butyl N-{[4-({[4-(2-phenylethyl)piperidin-1- yl]carbonylamino}methyl)phenyl]methyl}carbamate |
| 36 | 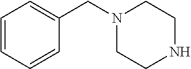 | Tert-butyl N-[(4-{[(4-benzylpiperazin-1- yl)carbonylamino]methyl}phenyl)methyl]carbamate |
| 37 | 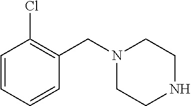 | Tert-butyl N-({4-[({4-[(2-chlorophenyl)methyl]piperazin-1- yl}carbonylamino)methyl]phenyl}methyl)carbamate |
| 38 | 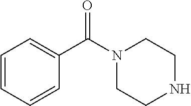 | Tert-butyl N-[(4-{[(4-benzoylpiperazin-1- yl)carbonylamino]methyl}phenyl)methyl]carbamate |
| 39 | 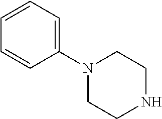 | Tert-butyl N-[(4-{[(4-phenylpiperazin-1- yl)carbonylamino]methyl}phenyl)methyl]carbamate |
| 40 | 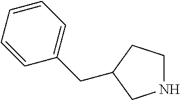 | Tert-butyl N-[(4-{[(3-benzylpyrrolidin-1- yl)carbonylamino]methyl}phenyl)methyl]carbamate |
| 41 | 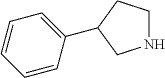 | Tert-butyl N-[(4-{[(3-phenylpyrrolidin-1- yl)carbonylamino]methyl}phenyl)methyl]carbamate |
| 42 | 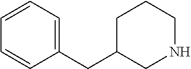 | Tert-butyl N-[(4-{[(3-benzylpiperidin-1- yl)carbonylamino]methyl}phenyl)methyl]carbamate |
| 43 | 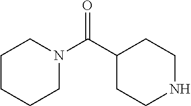 | Tert-butyl N-({4-[({4-[(piperidin-1-yl)carbonyl]piperidin-1- yl}carbonylamino)methyl]phenyl}methyl)carbamate |
| 44 | 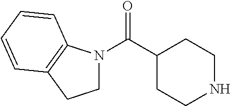 | Tert-butyl N-({4-[({4-[(2,3-dihydro-1H-indol-1- yl)carbonyl]piperidin-1-yl}carbonylamino)methyl]phenyl} methyl)carbamate |
| 45 | 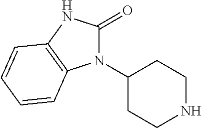 | Tert-butyl N-{[4-({[4-(2-oxo-2,3-dihydro-1H-1,3- benzodiazol-1-yl)piperidin-1-yl]carbonylamino} methyl)phenyl]methyl}carbamate |
| 46 | 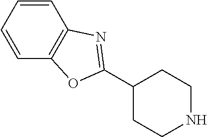 | Tert-butyl N-{[4-({[4-(1,3-benzoxazol-2-yl)piperidin-1- yl]carbonylamino}methyl)phenyl]methyl}carbamate |

### Intermediate 47

### Tert-butyl N-({4[({4-[(4-fluorophenyl)(hydroxy)methyl]piperidin-1-yl}carbonylamino)methyl]phenyl}methyl)carbamate

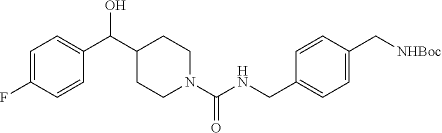 [SMILES: CC(C)(C)OC(=O)NCc1ccc(CNC(=O)N2CCC(C(O)c3ccc(F)cc3)CC2)cc1]

Intermediate 34 (46.0 mg, 0.10 mmol) was dissolved in MeOH (2 mL) and NaBH₄ (11.1 mg, 0.29 mmol) was added. The reaction mixture was stirred at room temperature for 1 h and quenched with water (1 mL). The solvents were removed in vacuo to give the crude title compound as a white solid which was used without further purification. LCMS: ES⁺ 372.5 [MH]⁺.

### Intermediate 48

### Tert-butyl N-({4[({1-[(3-fluorophenyl)methyl]piperidin-4-yl}formamido)methyl]phenyl}methyl)carbamate

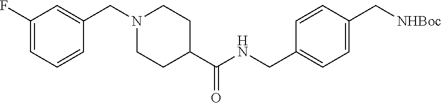 [SMILES: CC(C)(C)OC(=O)NCc1ccc(CNC(=O)C2CCN(Cc3cccc(F)c3)CC2)cc1]

1-[(3-Fluorophenyl)methyl]piperidine-4-carboxylic acid (350 mg, 1.28 mmol) was dissolved in THF (2 mL) and cooled to 0° C. DIPEA (0.46 mL, 2.66 mmol) and HBTU (480 mg, 1.27 mmol) were added and the reaction mixture was stirred for 20 min. Tert-butyl N-{[4-(aminomethyl)phenyl]methyl}carbamate (300 mg, 1.27 mmol) was added and the reaction mixture was warmed to room temperature over 21 h. The solvents were removed in vacuo and the residue was partitioned between EtOAc (25 mL) and water (15 mL). The organic fraction was washed with sat aq NH₄Cl (20 mL), 1 M aq Na₂CO₃ (20 mL) and brine (20 mL), dried (MgSO₄) and concentrated in vacuo. The residue was purified by column chromatography to give the title compound (380 mg, 66%). LCMS: purity 100%, ES⁺ 456.8 [MH]⁺.

### Intermediate 49

### General Procedure B

### 4-Benzyl-N-[(4-cyano-3-fluorophenyl)methyl]piperidine-1-carboxamide

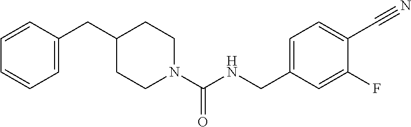 [SMILES: N#Cc1ccc(CNC(=O)N2CCC(Cc3ccccc3)CC2)cc1F]

Intermediate 8 (620 mg, 2.35 mmol), Intermediate 6 (970 mg, 2.35 mmol) and DIPEA (1.17 mL, 7.05 mmol) were dissolved in DMF (20 mL) and stirred at room temperature for 1 h. Further Intermediate 6 (243 mg, 0.59 mmol) was added, the reaction mixture was stirred for 4 h and the solvents were removed in vacuo. The residue was dissolved in EtOAc (50 mL), washed with 1 M aq HCl (20 mL), 1 M aq Na₂CO₃ (20 mL) and brine (30 mL) and concentrated in vacuo. The residue was purified by column chromatography to give the title compound (433 mg, 52%) as a yellow oil. LCMS: ES⁺ 352.6 [MH]⁺.

### Intermediates 50-55

Intermediates 50-55 were prepared similarly to General Procedure B using Intermediates 6 and 7; see Table 2 below.

TABLE 2
Coupling of amines with activated ureas

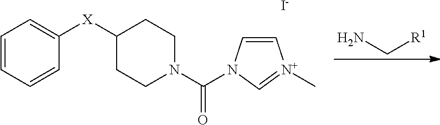

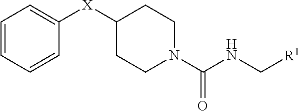

| Int | X | 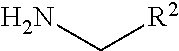 | Intermediate Name | Yield |
|---|---|---|---|---|
| 50 | bond | 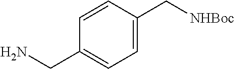 [SMILES: CC(C)(C)OC(=O)NCc1ccc(CN)cc1] | Tert-butyl N-[(4-{[(4-phenylpiperidin-1-yl) carbonylamino]methyl}phenyl)methyl]carbamate | 16% |
| 51 | CH₂ | 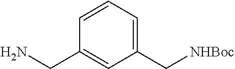 [SMILES: CC(C)(C)OC(=O)NCc1cccc(CN)c1] | Tert-butyl N-[(3-{[(4-benzylpiperidin-1-yl) carbonylamino]methyl}phenyl)methyl]carbamate | 31% |
| 52 | CH₂ | 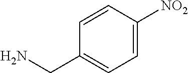 [SMILES: NCc1ccc([N+](=O)[O-])cc1] | 4-Benzyl-N-[(4-nitrophenyl)methyl]piperidine-1- carboxamide | 92% |
| 53 | CH₂ | 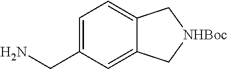 | Tert-butyl 5-{[(4-benzylpiperidin-1-yl)carbonyl amino]methyl}-2,3-dihydro-1H-isoindole-2- carboxylate | 82% |
| 54 | CH₂ | 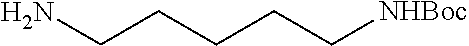 [SMILES: CC(C)(C)OC(=O)NCCCCCN] | Tert-butyl N-{5-[(4-benzylpiperidin-1-yl)carbonyl amino]pentyl}carbamate | 63% |
| 55 | CH₂ | 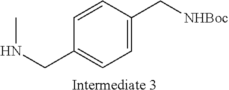 | Tert-butyl N-[(4-{[(4-benzylpiperidin-1-yl) carbonyl(methyl)amino]methyl}phenyl)methyl] carbamate | Used crude |

### Intermediate 56

### N-[(4-Aminophenyl)methyl]-4-benzylpiperidine-1-carboxamide

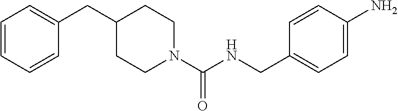 [SMILES: Nc1ccc(CNC(=O)N2CCC(Cc3ccccc3)CC2)cc1]

Intermediate 52 (390 mg, 1.10 mmol) was dissolved in AcOH (10 mL) and zinc dust (706 mg, 11.0 mmol) was added. The reaction mixture was stirred for 18 h and poured into 1 M aq Na₂CO₃ (150 mL). The solution was basified to pH 10 with NaOH and extracted with EtOAc (3×100 mL). The combined organic fractions were dried (MgSO₄) and the solvents removed in vacuo. The residue was purified by column chromatography to give the title compound (183 mg, 51%) as a colourless gum. LCMS: purity 100%, ES⁺ 324.7 [MH]⁺.

### Intermediate 57

### Tert-butyl N-[[(4-{[(4-benzylpiperidin-1-yl)carbonylamino]methyl}phenyl)amino]({[(tert-butoxy)carbonyl]imino})methyl]carbamate

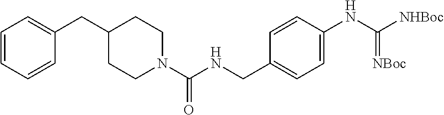

Intermediate 56 (132 mg, 0.41 mmol) was dissolved in DMF (5 mL) and tert-butyl N-[(1E)-{[(tert-butoxy)carbonyl]imino}(1H-pyrazol-1-yl)methyl]carbamate (127 mg, 0.41 mmol) was added. The reaction mixture was stirred for 6 d and the solvents were removed in vacuo. The residue was dissolved in EtOAc (50 mL), washed with 1 M aq Na₂CO₃ (3×50 mL), dried (MgSO₄) and the solvents were removed in vacuo. The residue was purified by column chromatography to give the title compound (231 mg, 96%) as a colourless gum. LCMS: purity 100%, ES⁺ 566.8 [MH]⁺.

### Intermediates 58

### Tert-butyl 5-({[4-(pyridin-4-ylmethyl)piperidin-1-yl]carbonylamino}methyl)-2,3-dihydro-1H-isoindole-2-carboxylate

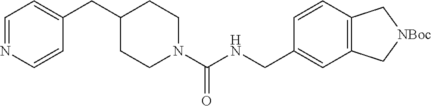 [SMILES: CC(C)(C)OC(=O)N1Cc2ccc(CNC(=O)N3CCC(Cc4ccncc4)CC3)cc2C1]

Intermediate 58 was prepared similarly to Intermediate 26, using tert-butyl 5-(aminomethyl)-2,3-dihydro-1H-isoindole-2-carboxylate instead of tert-butyl N-{[4-(aminomethyl)phenyl]methyl}carbamate, to give the title compound (86 mg, 13%).

LCMS: ES⁺ 451.7 [MH]⁺.

### Intermediates 59

### Tert-butyl 5-({[4-(4-fluorophenoxy)piperidin-1-yl]carbonylamino}methyl)-2,3-dihydro-1H-isoindole-2-carboxylate

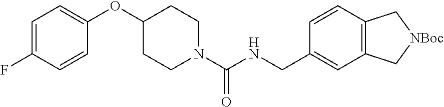 [SMILES: CC(C)(C)OC(=O)N1Cc2ccc(CNC(=O)N3CCC(Oc4ccc(F)cc4)CC3)cc2C1]

Intermediate 59 was prepared similarly to Intermediate 28, using tert-butyl 5-(aminomethyl)-2,3-dihydro-1H-isoindole-2-carboxylate instead of tert-butyl N-{[4-(aminomethyl)phenyl]methyl}carbamate, to give the crude title compound which was used without further purification. LCMS: ES⁺ 470.6 [MH]⁺.

### Intermediate 60

### 4-Benzyl-N-[(4-cyanophenyl)methyl]piperidine-1-carboxamide

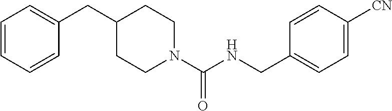 [SMILES: N#Cc1ccc(CNC(=O)N2CCC(Cc3ccccc3)CC2)cc1]

4-(Aminomethyl)benzonitrile hydrochloride (6.79 g, 40.3 mmol) and DIPEA (20.7 mL, 125 mmol) were dissolved in DMF (100 mL) and a solution of CDI (7.19 g, 44.3 mmol) in DMF (50 mL) was added. The reaction mixture was stirred for 2 h, benzylpiperidine (7.85 mL, 44.3 mmol) was added drop-wise and the reaction mixture was stirred for 16 h. The solvents were removed in vacuo and the residue was dissolved in EtOAc (150 mL) and washed with 1 M aq HCl (2×100 mL), 1 M aq Na₂CO₃ (100 mL) and brine and the solvents were removed in vacuo to give the title compound (12.9 g, 96%) as a yellow oil which was used without further purification. LCMS: ES⁺ 334.1 [MH]⁺.

### Intermediate 61

### N-[(4-Cyanophenyl)methyl]-4-(pyridin-4-ylmethyl)piperidine-1-carboxamide

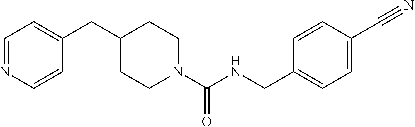 [SMILES: N#Cc1ccc(CNC(=O)N2CCC(Cc3ccncc3)CC2)cc1]

CDI (90.9 mg, 0.56 mmol) was dissolved in DCM (5 mL) at 0° C. and 4-aminomethyl-benzonitrile (90.0 mg, 0.53 mmol) and DIPEA (72.4 mg, 0.56 mmol) were added. The reaction mixture was warmed to room temperature over 1 h. The reaction mixture was cooled to 0° C. and a solution of 4-piperidin-4-ylmethyl pyridine dihydrochloride (139 mg, 0.56 mmol) and DIPEA (217 mg, 1.68 mmol) in DCM (5 mL) was added. The reaction mixture was stirred for 16 h and concentrated in vacuo. The residue was purified by HPLC to give the title compound (139 mg, 78%) as a colourless solid. LCMS: purity 100%, ES⁺ 335.6 [MH]⁺.

### Intermediate 62

### N-[(4-Cyanophenyl)methyl]-3-phenylpyrrolidine-1-carboxamide

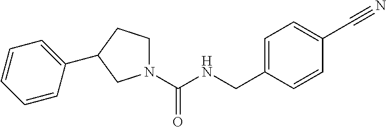 [SMILES: N#Cc1ccc(CNC(=O)N2CCC(c3ccccc3)C2)cc1]

CDI (441 mg, 2.72 mmol) was dissolved in DCM (25 mL) and cooled to 0° C. 3-Phenylpyrrolidine HCl (0.50 g, 2.72 mmol) and DIPEA (0.47 mL, 2.72 mmol) were added and the reaction mixture was warmed to room temperature and stirred for 24 h. The product was triturated with water (15 mL) and the solids collected by filtration. The residue was dissolved in MeCN (10 mL), iodomethane (0.68 mL, 10.9 mmol) was added and the reaction mixture was stirred for 3 d. The reaction mixture was concentrated in vacuo and re-dissolved in DMF (10 mL) and 4-aminomethylbenzonitrile HCl (0.37 g, 2.17 mmol) and DIPEA (0.72 mL, 4.17 mmol) were added. The reaction mixture was stirred for 48 h, concentrated in vacuo and partitioned between EtOAc (100 mL) and water (70 mL). The organic phase was washed with 1M aq HCl (30 mL), sat aq NaHCO₃ (30 mL), dried (MgSO₄) and concentrated in vacuo. The residue was triturated with ether (10 mL) to give the title compound (0.47 g, 71%) as a beige solid. LCMS: ES⁺ 306 [MH]^{+#}.

### Intermediate 63

### 4-Benzyl-N-[(4-cyanophenyl)methyl]piperazine-1-carboxamide

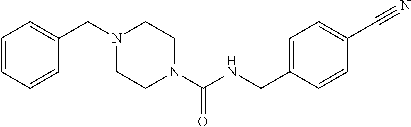 [SMILES: N#Cc1ccc(CNC(=O)N2CCN(Cc3ccccc3)CC2)cc1]

Intermediate 63 was prepared similarly to General Procedure A using 1-benzylpiperazine (0.77 mL, 4.44 mmol) and 4-aminomethylbenzonitrile HCl (0.75 g, 4.44 mmol) to give the title compound (1.22 g, 82%) as a yellow oil. LCMS: ES⁺ 335.2 [MH]^{+#}.

### Intermediate 64

### 4-[(Methylamino)methyl]benzonitrile

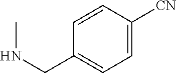

Methylamine (40% wt in H₂O, 25 mL, 322 mmol) was added to a solution of 4-(bromomethyl)benzonitrile (4.00 g, 20.4 mmol) in EtOH (40 mL) and the reaction mixture was stirred for 20 h. The reaction mixture was concentrated in vacuo and the residue purified by column chromatography to give the title compound (1.10 g, 37%) as a yellow oil. LCMS: ES⁺ 147.1 [MH]⁺.

### Intermediate 65

### 4-Benzyl-N-[(4-cyanophenyl)methyl]-N-methylpiperidine-1-carboxamide

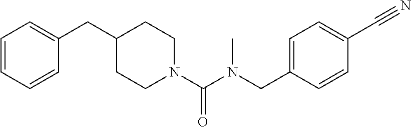 [SMILES: CN(Cc1ccc(C#N)cc1)C(=O)N1CCC(Cc2ccccc2)CC1]

The title compound was prepared similarly to General Procedure B using Intermediate 6 (1.80 g, 4.38 mmol) and Intermediate 64 (0.77 g, 5.27 mmol) to give the title compound (1.41 mg, 93%) as a yellow oil. LCMS: ES⁺ 348.1 [MH]^{+#}.

### Intermediate 66

### 1-Benzyl-N-[(4-cyanophenyl)methyl]piperidine-4-carboxamide

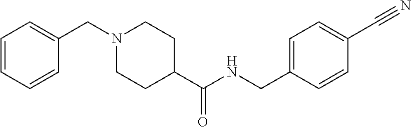 [SMILES: N#Cc1ccc(CNC(=O)C2CCN(Cc3ccccc3)CC2)cc1]

To a solution of 1-benzylpiperidine-4-carboxylic acid (1.00 g, 4.56 mmol) in DCM (50 mL) were added 1-(3-dimethylaminopropyl)-3-ethyl carbodiimide hydrochloride (0.90 g, 4.55 mmol) and hydroxybenzotriazole hydrate (0.70 g, 4.56 mmol) and the reaction mixture was stirred for 15 min. The reaction was cooled to 0° C. and 4-aminomethylbenzonitrile hydrochloride (0.77 g, 4.56 mmol) and DIPEA (1.58 mL, 9.13 mmol) were added and the reaction mixture was stirred for 20 h and concentrated in vacuo. The product was partitioned between EtOAc (70 mL) and sat aq NaHCO₃ (20 mL), and the aqueous phase extracted with EtOAc (50 mL). The organic fraction was washed with brine (50 mL), dried (MgSO₄) and concentrated in vacuo to give the title compound (1.34 g, 88%) as a yellow solid.

LCMS: ES⁺ 334.1 [MH]^{+#}.

### Intermediate 67

### 4-Benzyl-N-[(4-cyano-2-fluorophenyl)methyl]piperidine-1-carboxamide

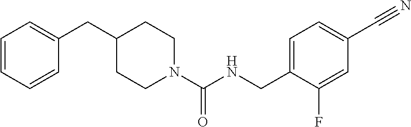 [SMILES: N#Cc1ccc(CNC(=O)N2CCC(Cc3ccccc3)CC2)c(F)c1]

The title compound was prepared similarly to General Procedure B using Intermediate 6 (0.76 g, 2.68 mmol) and 4-(aminomethyl)-3-fluoro-benzonitrile HCl (0.50 g, 2.68 mmol) to give the title compound (482 mg, 51%) as a yellow solid.

LCMS: ES⁺352.3 [MH]^{+#}.

### Intermediate 68

### N-[(4-Cyanophenyl)methyl]-4-[(3-methoxyphenyl)methyl]piperidine-1-carboxamide

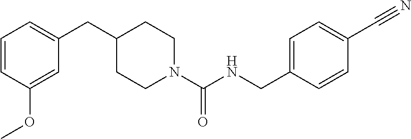 [SMILES: COc1cccc(CC2CCN(C(=O)NCc3ccc(C#N)cc3)CC2)c1]

CDI (613 mg, 3.78 mmol) was dissolved in DCM (30 mL) and cooled to 0° C. 3-Methoxybenzyl-4-piperidine (1.00 g, 3.78 mmol) and DIPEA (0.65 mL, 3.78 mmol) were added and the reaction mixture was warmed to room temperature and stirred for 3 d. The reaction mixture was concentrated in vacuo and partitioned between EtOAc (50 mL) and water (50 mL). The aqueous phase was washed with EtOAc (2×50 mL) and the combined organic fractions were washed with water (50 mL), dried (MgSO₄) and concentrated in vacuo. The residue was dissolved in MeCN (40 mL), iodomethane (1.17 mL, 18.9 mmol) was added and the reaction mixture stirred for 4 d. The reaction mixture was concentrated in vacuo and re-dissolved in DMF (22 mL) and 4-aminomethylbenzonitrile HCl (0.64 g, 3.78 mmol) and DIPEA (1.31 mL, 7.56 mmol) were added. The reaction mixture was stirred for 24 h, concentrated in vacuo and partitioned between EtOAc (100 mL) and water (50 mL). The organic phase was washed with water (2×50 mL) and brine (50 mL), dried (MgSO₄) and concentrated in vacuo to give the title compound (1.16 g, 84%) as a pale yellow oil.

LCMS: ES⁺ 364.0 [MH]^{+#}.

### Intermediate 69

### N-[(4-Cyanophenyl)methyl]-4-[(3-hydroxyphenyl)methyl]piperidine-1-carboxamide

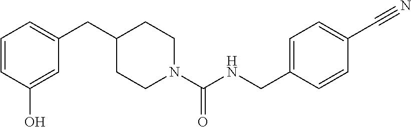 [SMILES: N#Cc1ccc(CNC(=O)N2CCC(Cc3cccc(O)c3)CC2)cc1]

To a solution of Intermediate 68 (314 mg, 0.86 mmol) in DCM (15 mL) at −78° C. was added BBr₃ (0.35 mL, 3.44 mmol) in DCM (5 mL). The reaction mixture was stirred for 1 h at −78° C. and then warmed gradually to room temperature and stirred for a further 2 h. The mixture was quenched by the addition of sat aq NH₄Cl (20 mL) and water (20 mL) and extracted with EtOAc (2×50 mL). The combined organic fractions were washed with brine (50 mL), dried (MgSO₄) and concentrated in vacuo to give the crude title compound as a white foam (254 mg, 85%) which was used without further purification. LCMS: ES⁻ 348 [M-H]^{-#}.

### Intermediate 70

### 4-Benzyl-N-(prop-2-yn-1-yl)piperidine-1-carboxamide

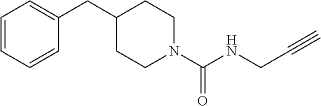 [SMILES: C#CCNC(=O)N1CCC(Cc2ccccc2)CC1]

The title compound was prepared similarly to General Procedure B using Intermediate 6 (2.00 g, 4.86 mmol) and propargylamine (0.31 mL, 4.86 mmol) to give the title compound (1.10 g, 88%) as a white solid. LCMS: ES⁺ 257.0 [MH]⁺.

### Intermediate 71

### 2-N-Benzyl-5-iodopyridine-2,4-diamine

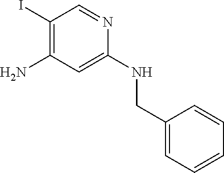 [SMILES: Nc1cc(NCc2ccccc2)ncc1I]

2-Chloro-5-iodopyridin-4-amine (1.78 g, 7.00 mmol) was dissolved in DMA (20 mL). 4-Methoxybenzylamine (4.57 mL, 35.0 mmol) and K₂CO₃ (2.90 g, 21.0 mmol) were added and the reaction mixture was heated using a Biotage microwave at 190° C. for 2 h. The solvents were removed in vacuo and the residue was purified by column chromatography to give the title compound (0.67 g, 27%) as a yellow solid. LCMS: ES⁺ 355.9 [MH]⁺.

### Intermediate 72

### 4-Benzyl-N-{[6-(benzylamino)-1H-pyrrolo[3,2-c]pyridin-2-yl]methyl}piperidine-1-carboxamide

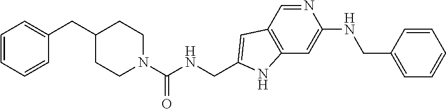 [SMILES: O=C(NCc1cc2cnc(NCc3ccccc3)cc2[nH]1)N1CCC(Cc2ccccc2)CC1]

Intermediate 70 (500 mg, 1.95 mmol) and Intermediate 71 (693 mg, 1.95 mmol) were dissolved in DMF (5 mL). Dichlorodi(triphenylpohsphino)palladium (68.7 mg, 0.10 mmol), copper iodide (11.1 mg, 0.06 mmol) and Et₃N (1.09 mL, 7.80 mmol) were added and the reaction was heated to 100° C. for 1.5 h. The reaction mixture was cooled to 50° C. and DBU (0.58 mL, 3.90 mmol) was added. The reaction mixture was heated to 50° C. for 30 min and cooled to room temperature. The reaction mixture was diluted with EtOAc (50 mL), washed with sat aq NH₄Cl (20 mL), water (20 mL), brine (20 mL), dried (MgSO₄) and concentrated in vacuo. The residue was purified by column chromatography to give the title compound (40.0 mg, 4.24%) as a white solid. LCMS: ES⁺ 484.1 [MH]⁺.

### Intermediate 73

### 4-Benzyl-N-[(4-cyanophenyl)methyl]piperidine-1-carbothioamide

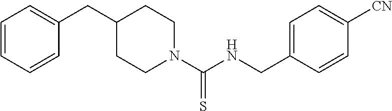 [SMILES: N#Cc1ccc(CNC(=S)N2CCC(Cc3ccccc3)CC2)cc1]

1,1′-Thiocarbonyldiimidazole (508 mg, 2.85 mmol) was suspended in THF (25 mL). 4-Benzylpiperidine (500 mg, 2.85 mmol) was added and the reaction mixture was stirred for 3 h. The solvents were removed in vacuo and the product dissolved in EtOAc (100 mL) and washed with water (100 mL), 10% aq citric acid (100 mL), sat aq NaHCO₃ (100 mL) and brine (100 mL), dried (MgSO₄) and the solvents were removed in vacuo. The residue was dissolved THF (10 mL) and MeI (1.06 mL, 16.8 mmol) was added. The reaction mixture was stirred for 48 h and concentrated in vacuo. The reaction mixture was re-dissolved in DMF (10 mL) and 4-aminomethylbenzonitrile HCl (283 mg, 1.68 mmol) and DIPEA (0.58 mL, 3.36 mmol) were added. The reaction mixture was stirred for 24 h, concentrated in vacuo and partitioned between EtOAc (100 mL) and water (50 mL). The organic phase was washed with water (2×50 mL) and brine (50 mL), dried (MgSO₄) and concentrated in vacuo to give the title compound (176 mg, 30%) as an orange oil.

LCMS: ES⁺ 350.0 [MH]^{+#}.

### Intermediate 74

### 4-{[(4-Benzylpiperidin-1-yl)carbonylamino]methyl}benzoate

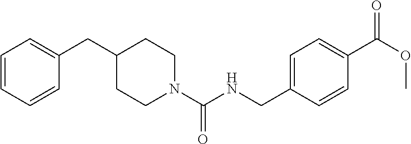 [SMILES: COC(=O)c1ccc(CNC(=O)N2CCC(Cc3ccccc3)CC2)cc1]

The title compound (1.22 g, 92%) was prepared similarly to Intermediate 49, using methyl 4-(aminomethyl)-benzoate hydrochloride instead of Intermediate 8, as a pale yellow solid. LCMS: ES⁺ 367.0 [MH]⁺.

### Intermediate 75

### 4-Benzyl-N-{[4-(methylcarbamoyl)phenyl]methyl}piperidine-1-carboxamide

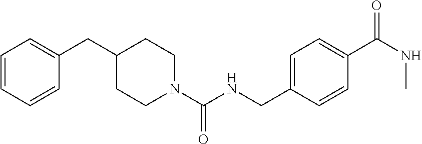 [SMILES: CNC(=O)c1ccc(CNC(=O)N2CCC(Cc3ccccc3)CC2)cc1]

4-{[(4-Benzyl-piperidine-1-carbonyl)-amino]-methyl}-benzoic acid methyl ester (1.22 g, 3.34 mmol) and LiOH monohydrate (0.70 g, 16.7 mmol) were dissolved in THF (15 mL) and water (15 mL) and stirred at 60° C. for 3 h. The organics were removed in vacuo and the solution diluted with water (5 mL) and acidified by the addition of 1 M aq HCl (10 mL). The mixture was extracted with DCM (2×30 mL). The combined organics were washed with water (30 mL), brine (30 mL) and concentrated in vacuo. The residue was dissolved in DMF (8 mL) and HBTU (0.48 g, 1.28 mmol) and DIPEA (0.58 mL, 3.48 mmol) were added followed by methylamine hydrochloride (0.16 g, 2.32 mmol). The reaction mixture was stirred at rt overnight.

The solvents were removed in vacuo. The product was suspended in DCM and sonicated, the ensuing solids were collected and washed with DCM to the title compound (0.34 g, 80.2%) as a white solid. LCMS: ES⁺ 366.0 [MH]⁺.

### Intermediate 76

### 6-(Aminomethyl)pyridine-3-carbonitrile

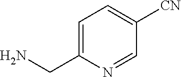

5-Cyano-2-methylpyridine (3.50 g, 29.6 mmol), AIBN (1.47 g, 9.00 mmol) and N-bromosuccinimide (5.60 g, 31.1 mmol) were dissolved in carbon tetrachloride (30 mL) and heated at reflux overnight. The reaction mixture was diluted with DCM (40 mL) and was washed sat aq NaHCO₃ (3×100 mL), dried (MgSO₄) and concentrated in vacuo. The product was purified by column chromatography. The residue was dissolved in THF (20 mL) and added to a stirring suspension of NaH (0.78 g, 19.5 mmol) in THF (20 mL) at 5° C. After 5 min a solution of di-tert-butyl iminodicarboxylate (3.87 g, 17.8 mmol) in THF (20 mL) was added and the reaction mixture warmed to room temperature and stirred overnight. The reaction mixture was concentrated in vacuo and partitioned between EtOAc (100 mL) and water (50 mL). The organic phase was washed with water (2×50 mL) and brine (50 mL), dried (MgSO₄) and the residue was purified by column chromatography. The residue was dissolved in MeOH (50 mL) and cooled to 0° C. HCl gas was bubbled through for 15 min and the resulting suspension stirred at room temperature for 3 h. The reaction mixture was concentrated in vacuo and the product triturated with hexane to afford the title compound (1.5 g, 97%) as a white solid. LCMS: ES⁺134.0 [MH]^{+#}.

### Intermediate 77

### 6-({[(4-Benzylpiperidin-1-yl)carbonyl]amino}methyl)pyridine-3-carboxamide

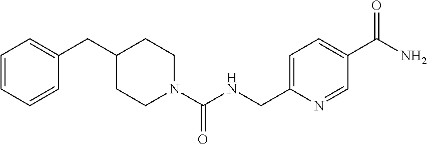 [SMILES: NC(=O)c1ccc(CNC(=O)N2CCC(Cc3ccccc3)CC2)nc1]

The title compound (1.30 g, 65%) was prepared similarly to Intermediate 49, using Intermediate 76 instead of Intermediate 8, as a pale yellow solid. LCMS: ES⁺ 353.0 [MH]^{+·}.

### Intermediate 78

### 4-Benzyl-N-[(5-cyanopyridin-2-yl)methyl]piperidine-1-carboxamide

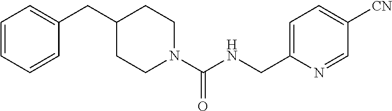 [SMILES: N#Cc1ccc(CNC(=O)N2CCC(Cc3ccccc3)CC2)nc1]

Intermediate 77 (353 mg, 1.00 mmol) and DIPEA (394 uL, 2.30 mmol) were dissolved in THF (15 mL), trifluoroacetic acid anhydride (153 uL, 1.15 mmol) was added and the reaction mixture stirred for 1 h. The reaction mixture was quenched with water (15 mL) and the organics removed in vacuo. The aqueous phase was extracted with EtOAc (2×30 mL), washed with 0.1 M aq HCl (30 mL), sat aq NaHCO₃ (30 mL), brine (20 mL), dried (MgSO₄) and concentrated in vacuo. The residue was purified by column chromatography to give the title compound (40.0 mg, 4.24%) as a yellow solid. LCMS: ES⁺ 335.0 [MH]^{+#}.

### Intermediate 79

### 4-Benzyl-N-[(6-cyanopyridin-3-yl)methyl]piperidine-1-carboxamide

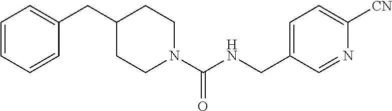 [SMILES: N#Cc1ccc(CNC(=O)N2CCC(Cc3ccccc3)CC2)cn1]

The title compound (1.30 g, 65%) was prepared similarly to Intermediate 49, using 5-aminomethylpyridine-2-carbonitrile hydrochloride instead of Intermediate 8, as a white solid. LCMS: ES⁺335.0 [MH]^{+#}.

### Example 1

### General Procedure C

### (4-{[(4-Benzylpiperidin-1-yl)carbonylamino]methyl}phenyl)methanaminium chloride

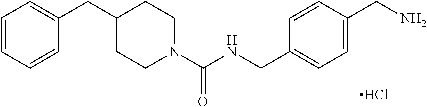 [SMILES: NCc1ccc(CNC(=O)N2CCC(Cc3ccccc3)CC2)cc1 ; Cl]

Intermediate 17 (365 mg, 0.83 mmol) was dissolved in dioxane (5 mL) and HCl (1.66 mL, 4 M in dioxane, 6.64 mmol) was added. The reaction mixture was stirred for 4 d and the precipitate was collected by filtration and washed with dioxane and ether to give the title compound (205 mg, 73%) as a yellow solid.

HRMS calculated for C₂₁H₂₈N₃O: 338.2232. found 338.2209. HPLC: Rf 9.49 min, 100% *.

### Examples 2-34

Examples 2-34 were prepared similarly to General Procedure C; see Table 3 below.

TABLE 3
Deprotection of intermediate Boc protected amines.

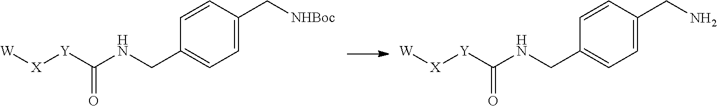

| | | | | | HRMS (ESI⁺)/ |
|---|---|---|---|---|---|
| Ex | Structure | Name | Int | Yield | LCMS/HPLC |
| 2 | 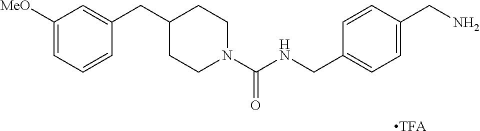 [SMILES: COc1cccc(CC2CCN(C(=O)NCc3ccc(CN)cc3)CC2)c1 ; "·TFA"] | 2,2,2-Trifluoroacetic acid; N-{[4-(aminomethyl) phenyl]methyl}-4-[(3- methoxyphenyl)methyl] piperidine-1-carboxamide | 18 | 43% | Calculated for C₂₂H₂₉N₃O₂: 367.225977, found 367.226437. HPLC: Rf 5.10 min, 100%. |
| 3 | 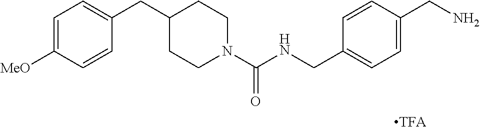 [SMILES: COc1ccc(CC2CCN(C(=O)NCc3ccc(CN)cc3)CC2)cc1 ; "·TFA"] | 2,2,2-Trifluoroacetic acid; N-{[4-(aminomethyl) phenyl]methyl}-4-[(4- methoxyphenyl)methyl] piperidine-1-carboxamide | 19 | 84% | Calculated for C₂₂H₂₉N₃O₂: 367.225977, found 367.226077. HPLC: Rf 5.08 min, 100%. |
| 4 | 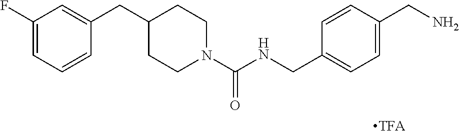 [SMILES: NCc1ccc(CNC(=O)N2CCC(Cc3cccc(F)c3)CC2)cc1 ; "·TFA"] | 2,2,2-Trifluoroacetic acid; N-{[4-(aminomethyl) phenyl]methyl}-4-[(3- fluorophenyl)methyl] piperidine-1-carboxamide | 20 | 45% | Calculated for C₂₁H₂₆FN₃O: 355.205991, found 355.207271. HPLC: Rf 5.16 min, 100%. |
| 5 | 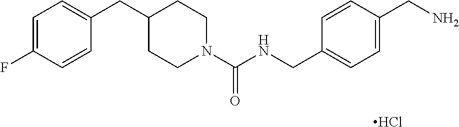 [SMILES: NCc1ccc(CNC(=O)N2CCC(Cc3ccc(F)cc3)CC2)cc1 ; Cl] | N-{[4-(Aminomethyl)phen- yl]methyl}-4-[(4- fluorophenyl)methyl]piper- idine-1-carboxamide hydrochloride | 21 | 60% | Calculated for C₂₁H₂₆FN₃O: 355.205991:, found 355.207661. HPLC: Rf 5.17 min, 100%. |
| 6 | 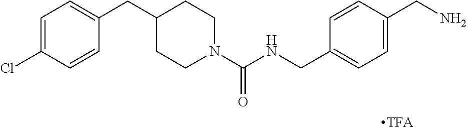 [SMILES: NCc1ccc(CNC(=O)N2CCC(Cc3ccc(Cl)cc3)CC2)cc1 ; "·TFA"] | 2,2,2-Trifluoroacetic acid; N-{[4-(aminomethyl) phenyl]methyl}-4-[(4- chlorophenyl)methyl] piperidine-1-carboxamide | 22 | 88% | Calculated for C₂₁H₂₆ClN₃O: 371.17644, found 371.1779. HPLC: Rf 5.46 min, 100%. |
| 7 | 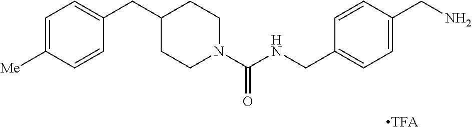 [SMILES: Cc1ccc(CC2CCN(C(=O)NCc3ccc(CN)cc3)CC2)cc1 ; "·TFA"] | 2,2,2-Trifluoroacetic acid; N-{[4-(aminomethyl) phenyl]methyl}-4-[(4- methylphenyl)methyl] piperidine-1-carboxamide | 23 | 48% | Calculated for C₂₂H₂₉N₃O: 351.231063, found 351.231643. HPLC: Rf 5.41 min, 99.6%. |
| 8 | 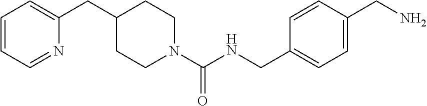 [SMILES: NCc1ccc(CNC(=O)N2CCC(Cc3ccccn3)CC2)cc1] | N-{[4-(Aminomethyl)phen- yl]methyl}-4-(pyridin- 2-ylmethyl)piperidine- 1-carboxamide | 24 | 38% | Calculated for C₂₂H₂₆N₄O: 338.210661, found 338.210661. HPLC: Rf 3.06 min, 100%. |
| 9 | 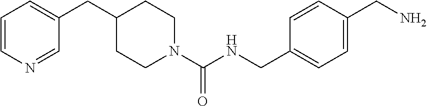 [SMILES: NCc1ccc(CNC(=O)N2CCC(Cc3cccnc3)CC2)cc1] | N-{[4-(Aminomethyl)phen- yl]methyl}-4-(pyridin- 3-ylmethyl)piperidine- 1-carboxamide | 25 | 51% | Calculated for C₂₀H₂₆N₄O: 338.210661, found 338.210671. HPLC: Rf 3.10 min, 100%. |
| 10 | 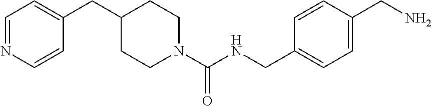 [SMILES: NCc1ccc(CNC(=O)N2CCC(Cc3ccncc3)CC2)cc1] | N-{[4-(Aminomethyl)phen- yl]methyl}-4-(pyridin- 4-ylmethyl)piperidine- 1-carboxamide | 26 | 35% | Calculated for C₂₀H₂₆N₄O: 338.210661, found 338.209881. HPLC: Rf 3.11 min, 100%. |
| 11 | 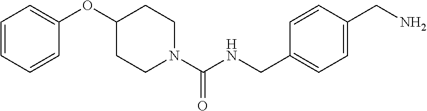 [SMILES: NCc1ccc(CNC(=O)N2CCC(Oc3ccccc3)CC2)cc1] | N-{[4-(Aminomethyl)phen- yl]methyl}-4- phenoxypiperidine- 1-carboxamide | 27 | 81% | Calculated for C₂₀H₂₅N₃O₂: 339.194677, found 339.194597. HPLC: Rf 4.71 min, 100%. |
| 12 | 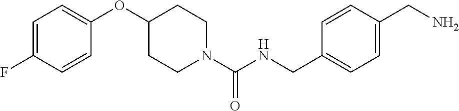 [SMILES: NCc1ccc(CNC(=O)N2CCC(Oc3ccc(F)cc3)CC2)cc1] | N-{[4-(Aminomethyl)phen- yl]methyl}-4-(4- fluorophenoxy)piperidine- 1-carboxamide | 28 | 84% | Calculated for C₂₀H₂₄FN₃O₂: 357.185255, found 357.187025. HPLC: Rf 4.77 min, 99.7%. |
| 13 | 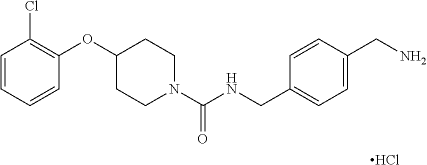 [SMILES: NCc1ccc(CNC(=O)N2CCC(Oc3ccccc3Cl)CC2)cc1 ; Cl] | N-{[4-(Aminomethyl)phen- yl]methyl}-4-(2- chlorophenoxy)piperidine- 1-carboxamide hydrochloride | 29 | 95% | Calculated for C₂₀H₂₄ClN₃O₂: 373.155705, found 373.156445. HPLC: Rf 5.01 min, 99.3%. |
| 14 | 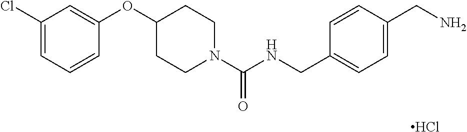 [SMILES: NCc1ccc(CNC(=O)N2CCC(Oc3cccc(Cl)c3)CC2)cc1 ; Cl] | N-{[4-(Aminomethyl)phen- yl]methyl}-4-(3- chlorophenoxy)piperidine- 1-carboxamide hydrochloride | 31 | 72% | Calculated for C₂₀H₂₄ClN₃O₂: 373.155705, found 373.156925. HPLC: Rf 5.18 min, 98.2%. |
| 15 | 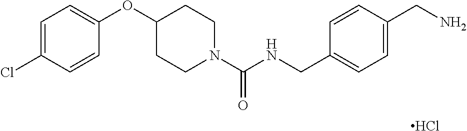 [SMILES: NCc1ccc(CNC(=O)N2CCC(Oc3ccc(Cl)cc3)CC2)cc1 ; Cl] | N-{[4-(Aminomethyl)phen- yl]methyl}-4-(4- chlorophenoxy)piperidine- 1-carboxamide hydrochloride | 31 | 66% | Calculated for C₂₀H₂₄ClN₃O₂: 373.155705, found 373.156815. HPLC: Rf 5.15 min, 100%. |
| 16 | 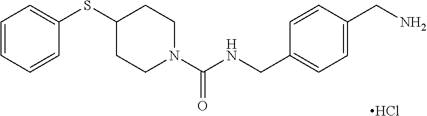 [SMILES: NCc1ccc(CNC(=O)N2CCC(Sc3ccccc3)CC2)cc1 ; Cl] | N-{[4-(Aminomethyl)phen- yl]methyl}-4- (phenylsulfanyl)piperidine- 1-carboxamide hydrochloride | 32 | 66% | Calculated for C₂₀H₂₅N₃OS: 355.171833, found 355.173533. HPLC: Rf 4.99 min, 100%. |
| 17 | 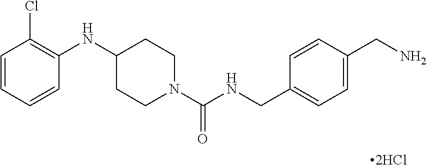 [SMILES: NCc1ccc(CNC(=O)N2CCC(Nc3ccccc3Cl)CC2)cc1 ; "·2HCl"] | N-{[4-(Aminomethyl)phen- yl]methyl}-4-[(2- chlorophenyl)amino]piper- idine-1-carboxamide dihydrochloride | 33 | 19% | Calculated for C₂₀H₂₅ClN₄O: 372.171689, found 372.173659. HPLC: Rf 4.96 min, 99.5%. |
| 18 | 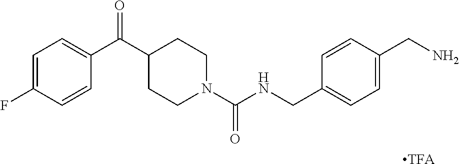 [SMILES: NCc1ccc(CNC(=O)N2CCC(C(=O)c3ccc(F)cc3)CC2)cc1 ; "·TFA"] | 2,2,2-Trifluoroacetic acid;- N-{[4-(aminomethyl) phenyl]methyl}-4-[(4- fluorophenyl)carbonyl] piperidine-1-carboxamide | 34 | 76% | Calculated for C₂₁H₂₄FN₃O₂: 369.185255, found 369.185265. HPLC: Rf 4.61 min, 100%. |
| 19 | 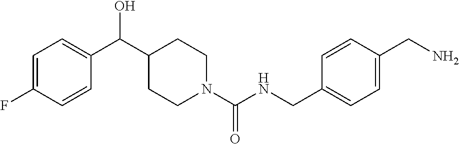 [SMILES: NCc1ccc(CNC(=O)N2CCC(C(O)c3ccc(F)cc3)CC2)cc1] | N-{[4-(Aminomethyl)phen- yl]methyl}-4-[(4- fluorophenyl)(hydroxy)meth- yl] piperidine-1- carboxamide | 47 | 79% | Calculated for C₂₁H₂₆FN₃O₂: 371.200905, found 371.200235. HPLC: Rf 4.27 min, 100%. |
| 20 | 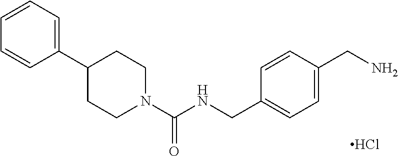 [SMILES: NCc1ccc(CNC(=O)N2CCC(c3ccccc3)CC2)cc1 ; Cl] | N-{[4-(Aminomethyl)phen- yl]methyl}-4- phenylpiperidine-1- carboxamide hydrochloride | 50 | 35% | Calculated for C₂₀H₂₅N₃O: 323.1998, found 323.2000. HPLC: Rf 4.78 min, 100%. |
| 21 | 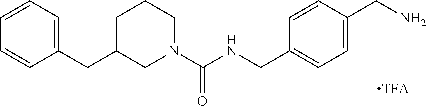 [SMILES: NCc1ccc(CNC(=O)N2CCCC(Cc3ccccc3)C2)cc1] | 2,2,2-Trifluoroacetic acid; N-{[4-(aminomethyl) phenyl]methyl}-3- benzylpiperidine-1- carboxamide | 42 | 57% | Calculated for C21H27N3O: 337.215413, found 337.215723. HPLC: Rf 5.04 min, 100%. |
| 22 | 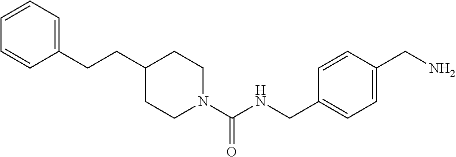 [SMILES: NCc1ccc(CNC(=O)N2CCC(CCc3ccccc3)CC2)cc1] | N-{[4-(Aminomethyl)phen- yl]methyl}-4-(2- phenylethyl)piperidine- 1-carboxamide | 35 | 55% | Calculated for C₂₂H₂₉N₃O: 351.231063, found 351.230673. HPLC: Rf 5.34 min, 99.6%. |
| 23 | 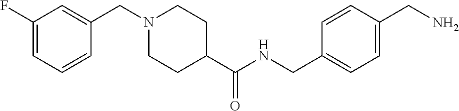 [SMILES: NCc1ccc(CNC(=O)C2CCN(Cc3cccc(F)c3)CC2)cc1] | N-{[4-(Aminomethyl)phen- yl]methyl}-1-[(3- fluorophenyl)methyl]piper- idine-4-carboxamide | 48 | 46% | Calculated for C₂₁H₂₆FN₃O: 355.2060, found 355.2060. HPLC: Rf 5.71 min, 100% *. |
| 24 | 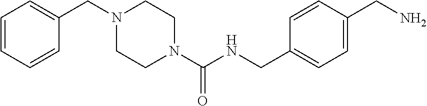 [SMILES: NCc1ccc(CNC(=O)N2CCN(Cc3ccccc3)CC2)cc1] | N-{[4-(Aminomethyl)phen- yl]methyl}-4- benzylpiperazine-1- carboxamide | 36 | 55% | Calculated for C₂₀H₂₆N₄O: 338.2107, found 338.2121. HPLC: Rf 5.28 min, 98.3% *. |
| 25 | 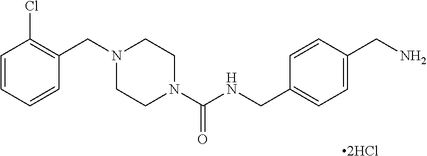 [SMILES: NCc1ccc(CNC(=O)N2CCN(Cc3ccccc3Cl)CC2)cc1 ; "·2HCl"] | N-{[4-(Aminomethyl)phen- yl]methyl]-4-[(2- chlorophenyl)meth- yl]piperazine-1- carboxamide di- hydrochloride | 37 | 78% | Calculated for C₂₀H₂₅ClN₄O: 372.171689, found 372.171999. HPLC: Rf 3.51 min, 100%. |
| 26 | 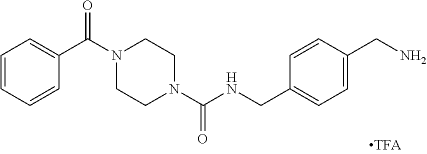 [SMILES: NCc1ccc(CNC(=O)N2CCN(C(=O)c3ccccc3)CC2)cc1] | 2,2,2-Trifluoroacetic acid; N-{[4-(aminomethyl) phenyl]methyl}-4- benzoylpiperazine-1- carboxamide | 38 | 41% | Calculated for C₂₀H₂₄N₄O₂: 352.189926, found 352.189326. HPLC: Rf 3.81 min, 100%. |
| 27 | 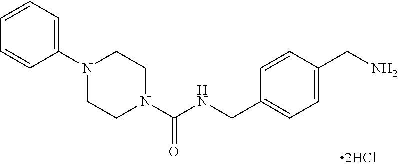 [SMILES: NCc1ccc(CNC(=O)N2CCN(c3ccccc3)CC2)cc1] | N-{[4-(Aminomethyl)phen- yl]methyl}-4-phenyl piperazine-1-carboxamide dihydrochloride | 39 | 90% | Calculated for C19H24N4O: 324.195011, found 324.194871. HPLC: Rf 4.85 min, 98.6% ***. |
| 28 | 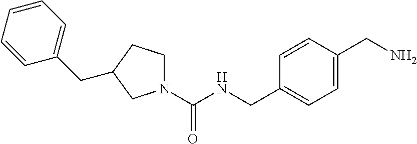 [SMILES: NCc1ccc(CNC(=O)N2CCC(Cc3ccccc3)C2)cc1] | N-{[4-(Aminomethyl)phen- yl]methyl}-3- benzylpyrrolidine-1- carboxamide | 40 | 50% | Calculated for C₂₀H₂₅N₃O: 323.199762, found 323.199772. HPLC: Rf 4.80 min, 100%. |
| 29 | 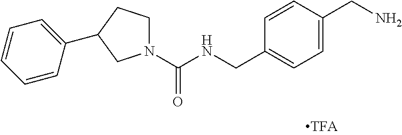 [SMILES: NCc1ccc(CNC(=O)N2CCC(c3ccccc3)C2)cc1] | 2,2,2-Trifluoroacetic acid; N-{[4-(aminomethyl) phenyl]methyl}-3- phenylpyrrolidine-1- carboxamide | 41 | 92% | LCMS: purity 100%, ES+ 310.7 [MH+]. HPLC: Rf 4.61 min, 100%. |
| 30 | 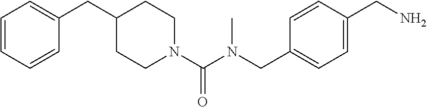 [SMILES: CN(Cc1ccc(CN)cc1)C(=O)N1CCC(Cc2ccccc2)CC1] | 2,2,2-Trifluoroacetic acid; N-{[4-(aminomethyl) phenyl]methyl}-4-benzyl-N- methylpiperidine-1- carboxamide | 55 | 35% | LCMS: purity 100%, ES+ 352.8 [MH+]. HPLC: Rf 5.29 min, 100%. |
| 31 | 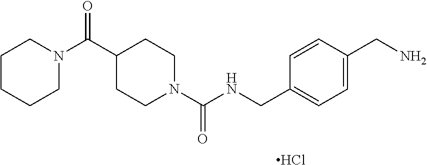 [SMILES: NCc1ccc(CNC(=O)N2CCC(C(=O)N3CCCCC3)CC2)cc1 ; Cl] | N-{[4-(Aminomethyl)phen- yl]methyl}-4- [(piperidin-1-yl)carbon- yl]piperidine-1- carboxamide hydrochloride | 43 | 73% | Calculated for C20H30N4O2: 358.236876, found 358.237466. HPLC: Rf 3.96 min, 99.6% **. |
| 32 | 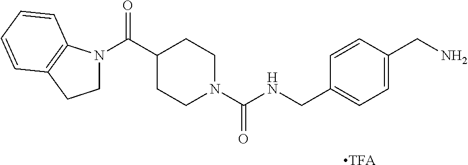 [SMILES: NCc1ccc(CNC(=O)N2CCC(C(=O)N3CCc4ccccc43)CC2)cc1 ; "·TFA"] | 2,2,2-Trifluoroacetic acid; N-{[4-(amino methyl)phenyl]methyl}-4- [(2,3-dihydro-1H- indol-1-yl)carbonyl]piper- idine-1-carboxamide | 44 | 80% | Calculated for C23H28N4O2: 392.221226, found 392.221436. HPLC: Rf 4.55 min, 100%. |
| 33 | 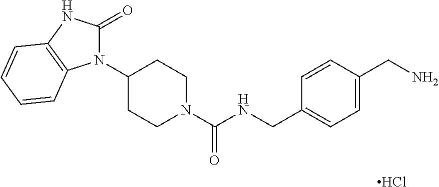 [SMILES: NCc1ccc(CNC(=O)N2CCC(n3c(=O)[nH]c4ccccc43)CC2)cc1 ; Cl] | N-{[4-(Aminomethyl)phen- yl]methyl}-4-(2-oxo- 2,3-dihydro-1H-1,3- benzodiazol-1- yl)piperidine-1-carboxa- mide hydrochloride | 45 | 83% | Calculated for C₂₁H₂₅N₅O₂: 379.200825, found 379.201015. HPLC: Rf 3.98 min, 100%. |
| 34 | 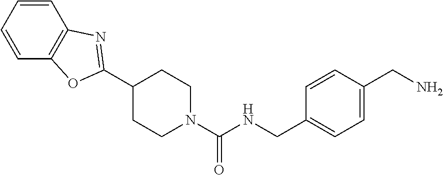 [SMILES: NCc1ccc(CNC(=O)N2CCC(c3nc4ccccc4o3)CC2)cc1] | N-{[4-(Aminomethyl)phen- yl]methyl}-4-(1,3- benzoxazol-2-yl)pipe- ridine-1-carboxamide | 46 | 30% | Calculated for C₂₁H₂₄N₄O₂: 364.189926, found 364.190236. HPLC: Rf 4.46 min, 99.8%. |

### Example 35

### N-{[4-(Aminomethyl)-3-fluorophenyl]methyl}-4-benzylpiperidine-1-carboxamide

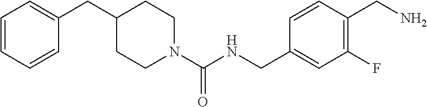 [SMILES: NCc1ccc(CNC(=O)N2CCC(Cc3ccccc3)CC2)cc1F]

Intermediate 49 (0.130 mg, 0.36 mmol) and CoCl₂ (47.0 mg, 0.36 mmol) were dissolved in MeOH (4 mL) and NaBH₄ (82.0 mg, 2.16 mmol) was added portion-wise. The reaction mixture was stirred at room temperature for 1 h, poured into 1 M aq Na₂CO₃ (25 mL) and extracted with EtOAc (2×25 mL). The combined organic fractions were washed with brine (50 mL) and the solvents were removed in vacuo. The residue was purified by HPLC (1% formic acid) and de-salted (K₂CO₃ in DCM) to give the title compound (20.0 mg, 16%) as a white solid. HRMS calculated for C₂₁H₂₆FN₃O: 355.205991. found 355.205651. HPLC: Rf 5.11 min, 98.5%.

### Example 36

### General Procedure D

### 4-Benzyl-N-[(4-carbamimidoylphenyl)methyl]piperidine-1-carboxamide

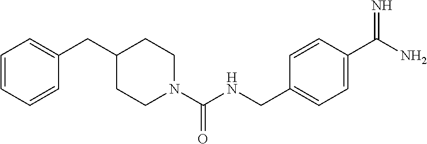 [SMILES: N=C(N)c1ccc(CNC(=O)N2CCC(Cc3ccccc3)CC2)cc1]

Intermediate 60 (6.00 g, 18.0 mmol) was dissolved in EtOH (150 mL), cooled to 0° C. and HCl (g) was bubbled through the solution for 45 min. The reaction mixture was warmed to room temperature over 16 h and the solvents were removed in vacuo. The residue was dissolved in 7 M NH₃ in MeOH (100 mL) and the reaction mixture was stirred for 64 h. The solvents were removed in vacuo and the residue was dissolved in boiling EtOH/MeOH (10:1), filtered, and concentrated in vacuo. The residue was purified by column chromatography to give the title compound (4.45 g, 71%) as a white solid. HRMS calculated for C₂₁H₂₆N₄O: 350.210662. found 350.211802. HPLC: Rf 5.11 min, 100%.

### Examples 37-47

Examples 37-47 were prepared similarly to General Procedure D; see Table 4 below.

TABLE 4
Preparation of amidines.

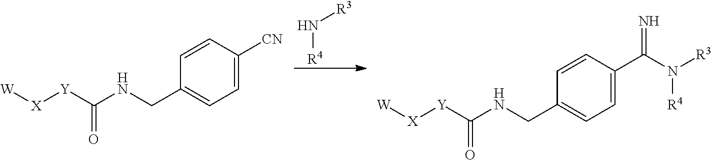

| | | | | | HRMS (ESI⁺)/ |
|---|---|---|---|---|---|
| Ex | Structure | Name | Int | Yield | LCMS/HPLC |
| 37 | 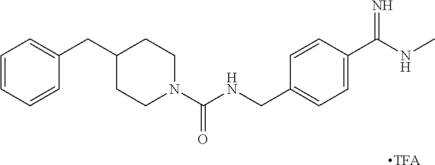 [SMILES: CNC(=N)c1ccc(CNC(=O)N2CCC(Cc3ccccc3)CC2)cc1 ; "·TFA"] | 2,2,2-Trifluoroacetic acid; 4-benzyl-N-{[4-(N- methylcarbamimidoyl)phen- yl]methyl} piperidine-1- carboxamide | 60 | 1% | Calculated for C₂₂H₂₈N₄O: 364.2263, found 364.2264. HPLC: Rf 5.20 min, 98.0%. |
| 38 | 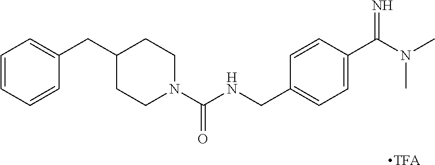 [SMILES: CN(C)C(=N)c1ccc(CNC(=O)N2CCC(Cc3ccccc3)CC2)cc1 ; "·TFA"] | 2,2,2-Trifluoroacetic acid; 4-benzyl-N-{[4- (N,N-dimethylcar- bamimidoyl)phen- yl]methyl} piperidine-1- carboxamide | 60 | 39% | Calculated for C₂₃H₃₀N₄O: 378.241962, found 378.243702. HPLC: Rf 5.41 min, 96.1%. |
| 39 | 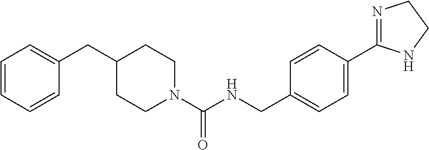 [SMILES: O=C(NCc1ccc(C2=NCCN2)cc1)N1CCC(Cc2ccccc2)CC1] | 4-Benzyl-N-{[4-(4,5-dihydro- 1H-imidazol-2- yl)phenyl]methyl}piperidine- 1-carboxamide | 60 | 7% | Calculated for C₂₃H₂₈N₄O: 376.226312, found 376.227352. HPLC: Rf 5.21 min, 99.2%. |
| 40 | 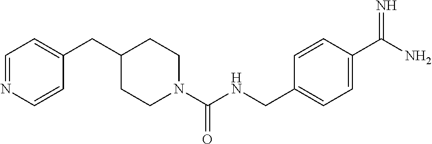 [SMILES: N=C(N)c1ccc(CNC(=O)N2CCC(Cc3ccncc3)CC2)cc1] | N-[(4-Carbamimidoylphen- yl)methyl]-4- (pyridin-4-ylmethyl)piperi- dine-1-carboxamide | 61 | 11% | Calculated for C₂₀H₂₅N₅O: 351.2059, found 351.2067. HPLC: Rf 3.89 min, 98.8%. |
| 41 | 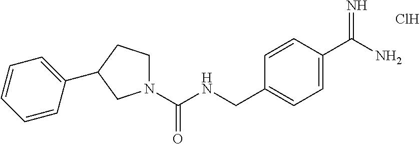 [SMILES: N=C(N)c1ccc(CNC(=O)N2CCC(c3ccccc3)C2)cc1 ; Cl] | N-[(4-Carbamimidoylphen- yl)methyl]-3- phenylpyrrolidine-1- carboxamide hydrochloride | 62 | 48% | LCMS: purity 100%, ES⁺ 322.9 [MH]⁺. HPLC: Rf 4.44 min, 100%. |
| 42 | 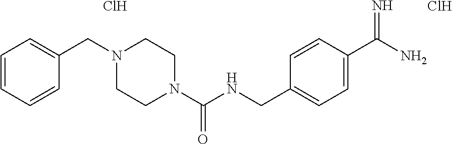 [SMILES: N=C(N)c1ccc(CNC(=O)N2CCN(Cc3ccccc3)CC2)cc1 ; Cl ; Cl] | 4-Benzyl-N-[(4- carbamimidoylphenyl)meth- yl]piperazine-1- carboxamide dihydrochloride | 63 | 15% | LCMS: purity 100%, ES⁺ 352.0 [MH]⁺. HPLC: Rf 3.03 min, 98%. |
| 43 | 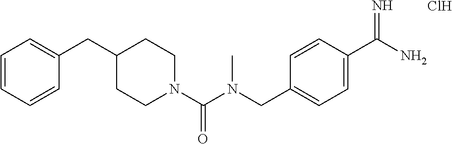 [SMILES: CN(Cc1ccc(C(=N)N)cc1)C(=O)N1CCC(Cc2ccccc2)CC1 ; Cl] | 4-Benzyl-N-[(4- carbamimidoylphenyl)methyl]-N- methylpiperidine-1-carboxamide hydrochloride | 65 | 8% | LCMS: purity 100%, ES⁺ 365.0 [MH]⁺. HPLC: Rf 5.15 min, 99%. |
| 44 | 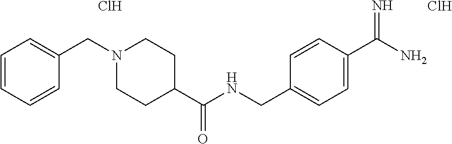 [SMILES: N=C(N)c1ccc(CNC(=O)C2CCN(Cc3ccccc3)CC2)cc1 ; Cl ; Cl] | 1-Benzyl-N-[(4- carbamimidoylphenyl)meth- yl]piperidine-4- carboxamide dihydrochloride | 66 | 12% | LCMS: purity 100%, ES⁺ 351.0 [MH]⁺. HPLC: Rf 3.13 min, 99%. |
| 45 | 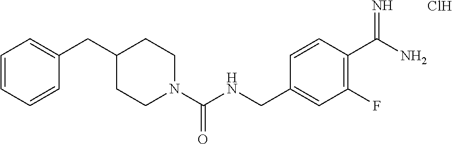 [SMILES: N=C(N)c1ccc(CNC(=O)N2CCC(Cc3ccccc3)CC2)cc1F ; Cl] | 4-Benzyl-N-[(4- carbamimidoyl-3- fluorophenyl)meth- yl]piperidine-1- carboxamide hydrochloride | 49 | 10% | LCMS: purity 100%, ES⁺ 369.0 [MH]⁺. HPLC: Rf 5.04 min, 97%. |
| 46 | 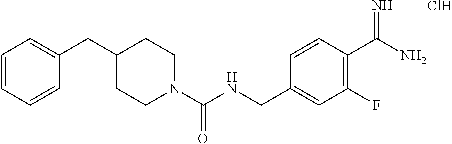 [SMILES: N=C(N)c1ccc(CNC(=O)N2CCC(Cc3ccccc3)CC2)cc1F ; Cl] | N-[(4-Carbamimidoylphen- yl)methyl]-4-[(3- hydroxyphenyl)meth- yl]piperidine-1- carboxamide hydrochloride | 67 | 68% | LCMS: purity 100%, ES⁺ 369.0 [MH]⁺. HPLC: Rf 5.01 min, 97%. |
| 47 | 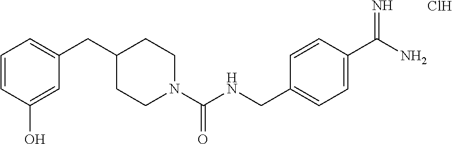 [SMILES: N=C(N)c1ccc(CNC(=O)N2CCC(Cc3cccc(O)c3)CC2)cc1 ; Cl] | N-[(4-Carbamimidoylphen- yl)methyl]-4-[(3- hydroxyphenyl)meth- yl]piperidine-1- carboxamide hydrochloride | 69 | 66% | LCMS: purity 100%, ES⁺ 367.0 [MH]⁺. HPLC: Rf 4.26 min, 96%. |
| 48 | 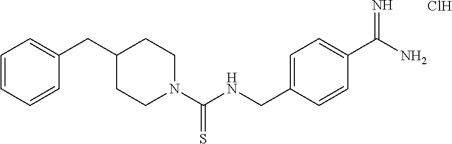 [SMILES: N=C(N)c1ccc(CNC(=S)N2CCC(Cc3ccccc3)CC2)cc1 ; Cl] | 4-Benzyl-N-[(4- carbamimidoylphen- yl)methyl]piperidine-1- carbothioamide hydrochloride | 73 | 14% | LCMS: purity 100%, ES⁺ 367.0 [MH]⁺. HPLC: Rf 5.41 min, 95%. |
| 49 | 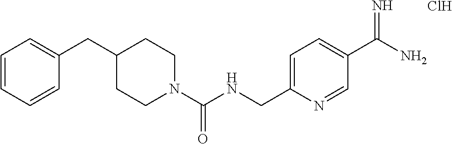 [SMILES: N=C(N)c1ccc(CNC(=O)N2CCC(Cc3ccccc3)CC2)nc1 ; Cl] | 4-Benzyl-N-[(5-carbamimidoyl- pyridin-2- yl)methyl]piperidine- 1-carboxamide hydrochloride | 78 | 26% | LCMS: purity 100%, ES⁺ 352.0 [MH]⁺ HPLC: Rf 4.67 min, 99%. |
| 50 | 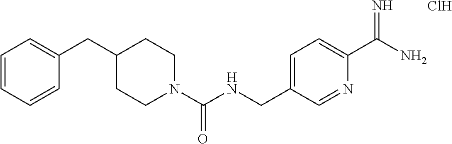 [SMILES: N=C(N)c1ccc(CNC(=O)N2CCC(Cc3ccccc3)CC2)cn1 ; Cl] | 4-Benzyl-N-[(6-carbam- imidoylpyridin-3- yl)methyl]piperidine- 1-carboxamide hydrochloride | 79 | 40% | LCMS: purity 100%, ES⁺ 352.0 [MH]⁺ HPLC: Rf 4.93 min, 99%. |

### Example 51

### 4-Benzyl-N-{[4-(1H-imidazol-2-yl)phenyl]methyl}piperidine-1-carboxamide

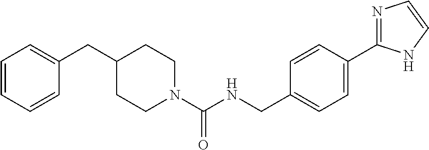 [SMILES: O=C(NCc1ccc(-c2ncc[nH]2)cc1)N1CCC(Cc2ccccc2)CC1]

Intermediate 10 (100 mg, 0.57 mmol) and DIPEA (0.28 mL, 1.71 mmol) were dissolved in DMF (5 mL) and Intermediate 6 (230 mg, 0.57 mmol) was added. The reaction mixture was stirred for 16 h and the solvents were removed in vacuo. The residue was dissolved in EtOAc (50 mL), washed with 1 M aq HCl (20 mL), 1 M aq Na₂CO₃ (20 mL) and brine (30 mL) and the solvents were removed in vacuo. The residue was purified by HPLC to give the title compound (61.9 mg, 29%) as a white solid. HRMS calculated for C₂₃H₂₆N₄O: 374.210661. found 374.211711. HPLC: Rf 5.30 min, 100%.

### Example 52

### N-(1H-1,3-Benzodiazol-6-ylmethyl)-4-benzylpiperidine-1-carboxamide

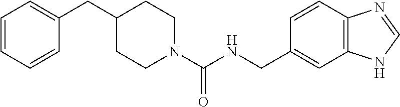 [SMILES: O=C(NCc1ccc2nc[nH]c2c1)N1CCC(Cc2ccccc2)CC1]

The title compound (4.06 mg, 7%) was prepared similarly to Example 48, using Intermediate 12 instead of Intermediate 10, as a white solid. HRMS calculated for C₂₁H₂₄N₄O: 348.195011. found 348.195601. HPLC: Rf 5.11 min, 100%.

### Example 53

### 4-Benzyl-N-{1H-pyrrolo[3,2-c]pyridin-2-ylmethyl}piperidine-1-carboxamide

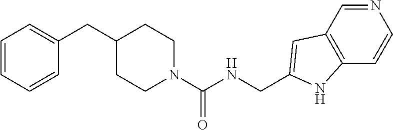 [SMILES: O=C(NCc1cc2cnccc2[nH]1)N1CCC(Cc2ccccc2)CC1]

The title compound (6.27 mg, 3%) was prepared similarly to Example 48, using 1H-pyrrolo[3,2-c]pyridin-2-ylmethanamine instead of Intermediate 10, as a white solid. HRMS calculated for C₂₁H₂₄N₄O: 348.195011. found 348.195711.

HPLC: Rf 5.18 min, 99.8%.

### Example 54

### 4-Benzyl-N-[(1-methyl-1H-1,3-benzodiazol-6-yl)methyl]piperidine-1-carboxamide

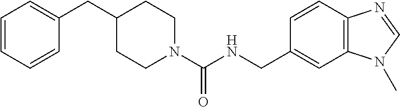 [SMILES: Cn1cnc2ccc(CNC(=O)N3CCC(Cc4ccccc4)CC3)cc21]

Example 49 (100 mg, 0.29 mmol) and Cs₂CO₃ (90.0 mg, 0.29 mmol) were dissolved in DMF (2 mL), iodomethane (18.0 μL, 0.29 mmol) was added and the mixture stirred for 3 h. The reaction mixture was diluted with MeOH and concentrated in vacuo. The residue was purified by reverse phase HPLC to give the title compound (21.1 mg, 20%) as a white solid. HRMS calculated for C₂₂H₂₆N₄₀: 362.210661. found 362.212241. HPLC: Rf 5.24 min, 99.3%.

### Example 55

### N-(1H-1,3-Benzodiazol-6-ylmethyl)-4-(4-fluorophenoxy)piperidine-1-carboxamide

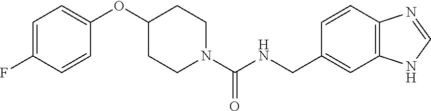 [SMILES: O=C(NCc1ccc2nc[nH]c2c1)N1CCC(Oc2ccc(F)cc2)CC1]

CDI (60.6 mg, 0.37 mmol) was dissolved in DMF (2.5 mL) and a solution of Intermediate 12 (50.0 mg, 0.34 mmol) in DMF (0.5 mL) was added. The reaction mixture was stirred for 1 h and a solution of 4-(4-fluorophenoxy)piperidine hydrochloride (86.6 mg, 0.37 mmol) and DIPEA (130 uL, 0.75 mmol) in DMF (3.0 mL) was added. The reaction mixture was stirred for 16 h and concentrated in vacuo. The residue was purified by column chromatography and HPLC to give the title compound (61.4 mg, 49%) as a white solid. HRMS calculated for C₂₀H₂₁FN₄O₂: 368.164854. found 368.164934. HPLC: Rf 4.83 min, 100%.

### Examples 56-60

Examples 56-60 were prepared similarly to Example 55, using the appropriate commercially available cyclic amine derivative instead of 4-(4-fluorophenoxy)piperidine; see Table 5 below.

TABLE 5
Urea formation from 1H-1,3-benzodiazol-6-ylmethanamine.

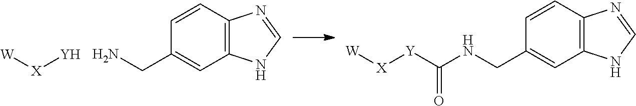

| Ex | Structure | Name | Yield | HRMS (ESI⁺)/HPLC |
|---|---|---|---|---|
| 56 | 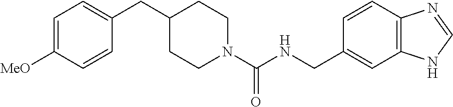 [SMILES: COc1ccc(CC2CCN(C(=O)NCc3ccc4nc[nH]c4c3)CC2)cc1] | N-(1H-1,3-Benzodiazol- 6-ylmethyl)-4-[(4- methoxyphenyl)methyl] piperidine-1- carboxamide | 56% | Calculated for C₂₂H₂₆N₄O₂: 378.205576, found 378.206696. HPLC: Rf 5.05 min, 100%. |
| 57 | 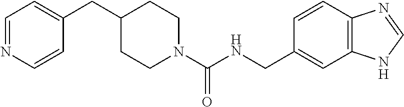 [SMILES: O=C(NCc1ccc2nc[nH]c2c1)N1CCC(Cc2ccncc2)CC1] | N-(1H-1,3-Benzodiazol-6- ylmethyl)-4-(pyridin-4- ylmethyl)piperidine-1- carboxamide | 3% | Calculated for C₂₀H₂₃N₅O: 349.19026, found 349.19074. HPLC: Rf 3.89 min, 100%. |
| 58 | 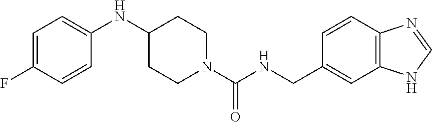 [SMILES: O=C(NCc1ccc2nc[nH]c2c1)N1CCC(Nc2ccc(F)cc2)CC1] | N-(1H-1,3-Benzodiazol-6- ylmethyl)-4-[(4- fluorophenyl)amino]piperidine- 1-carboxamide | 31% | Calculated for C₂₀H₂₂FN₅O: 367.180839, found 367.180389. HPLC: Rf 3.47 min, 99.6%. |
| 59 | 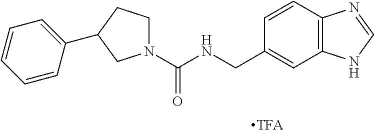 [SMILES: O=C(NCc1ccc2nc[nH]c2c1)N1CCC(c2ccccc2)C1] | 2,2,2-Trifluoroacetic acid; N-(1H-1,3- benzodiazol-5-ylmethyl)-3- phenylpyrrolidine-1- carboxamide | 33% | Calculated for C₁₉H₂₀N₄O: 320.163711, found 320.163991. HPLC: Rf 4.60 min, 100%. |
| 60 | 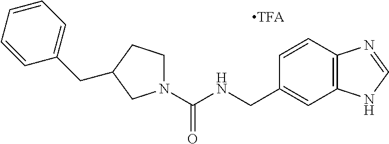 [SMILES: O=C(NCc1ccc2nc[nH]c2c1)N1CCC(Cc2ccccc2)C1] | 2,2,2-Trifluoroacetic acid; N-(1H-1,3- benzodiazol-5-ylmethyl)- 3-benzylpyrrolidine-1- carboxamide | 35% | Calculated for C₂₀H₂₂N₄O: 334.179361, found 334.179821. HPLC: Rf 4.82 min, 100%. |

### Example 61

### N-(1H-1,2,3-Benzotriazol-6-ylmethyl)-4-benzylpiperidine-1-carboxamide

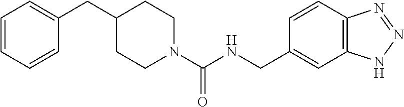 [SMILES: O=C(NCc1ccc2nn[nH]c2c1)N1CCC(Cc2ccccc2)CC1]

Intermediate 16 (50.0 mg, 0.30 mmol), Intermediate 6 (130 mg, 0.30 mmol) and DIPEA (0.15 mL, 0.91 mmol) were dissolved in DMF (3 mL) and stirred for 16 h. Further Intermediate 6 (65.0 mg, 0.15 mol) was added and the reaction mixture was stirred for 2 h and concentrated in vacuo. The residue was dissolved in EtOAc (200 mL), washed with 1M aq Na₂CO₃ (20 mL), sat aq NH₄Cl (20 mL) and brine (30 mL) and concentrated in vacuo. The residue was purified by reverse phase HPLC, dissolved in THF and water (4 mL, 1:1) and LiOH (excess) was added. The reaction mixture was heated at 50° C. for 3 h and the solvents were removed in vacuo. The residue was dissolved in EtOAc (30 mL), washed with water (20 mL) and brine (20 mL) and concentrated in vacuo. The residue was purified by reverse phase HPLC and de-salted (K₂CO₃ in DCM) to give the title compound (9.10 mg, 8%) as a white solid. HRMS calculated for C₂₀H₂₃N₅₀: 349.19026. found 349.19027. HPLC: Rf 5.84 min, 99.8%.

### Example 62

### N-[(2-Amino-1H-1,3-benzodiazol-6-yl)methyl]-4-benzylpiperidine-1-carboxamide

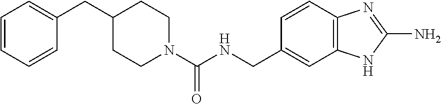 [SMILES: Nc1nc2ccc(CNC(=O)N3CCC(Cc4ccccc4)CC3)cc2[nH]1]

Intermediate 14 (40.0 mg, 0.25 mmol), Intermediate 6 (210 mg, 0.50 mmol) and DIPEA (120 μL, 0.75 mmol) were dissolved in DMF (3 mL) and the reaction mixture was stirred for 16 h. The solvents were removed in vacuo and the residue was diluted with EtOAc (30 mL), washed with water (20 mL), 1 M aq Na₂CO₃ (30 mL), and brine and the solvents were removed in vacuo. The residue was dissolved in THF/Water (1:1, 5 mL), an excess of LiOH was added and the reaction mixture was stirred for 16 h. The solvents were removed in vacuo and the residue was dissolved in EtOAc (30 mL), washed with water (20 mL) and brine (20 mL) and concentrated in vacuo. The residue was purified by HPLC (1% TFA), desalted (K₂CO₃ in DCM) and purified by column chromatography to give the title compound (9.13 mg, 10%) as a white solid. HRMS calculated for O₂₁H₂₅N₅O: 363.20591. found 363.20616. HPLC: Rf 5.29 min, 97.1%.

### Example 63

### 2,2,2-Trifluoroacetic acid; 4-benzyl-N-[(4-carbamimidamidophenyl)methyl]piperidine-1-carboxamide

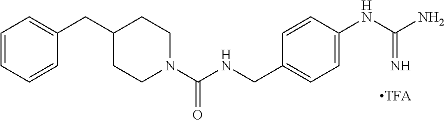 [SMILES: N=C(N)Nc1ccc(CNC(=O)N2CCC(Cc3ccccc3)CC2)cc1]

Intermediate 57 (230 mg, 0.41 mmol) was dissolved in DCM (5 mL), TFA (2 mL) was added and the reaction mixture was stirred for 3 h. The solvents were removed in vacuo and the residue was triturated with Et₂O and purified by HPLC (1% TFA) to give the title compound (98.0 mg, 50%) as a colourless gum. HRMS calculated for C₂₀H₂₅N₅O: 351.20591. found 351.20703. HPLC: Rf 5.23 min, 100%.

### Examples 64-68

Examples 64-68 were prepared similarly to Example 63; see Table 6 below.

TABLE 6
Deprotection of intermediate Boc protected amines.

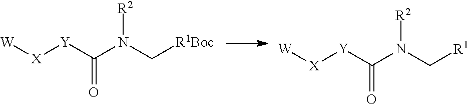

| Ex | Structure | Name | Int | Yield | HRMS (ESI⁺)/LCMS/HPLC |
|---|---|---|---|---|---|
| 64 | 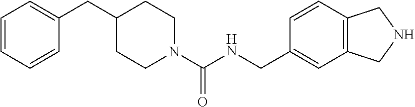 [SMILES: O=C(NCc1ccc2c(c1)CNC2)N1CCC(Cc2ccccc2)CC1] | 4-Benzyl-N-(2,3- dihydro-1H- isoindol-5-ylmeth- yl)piperidine-1- carboxamide | 53 | 64% | Calculated for C₂₂H₂₇N₃O: 349.215413, found 349.215873. HPLC: Rf 5.12 min, 100%. |
| 65 | 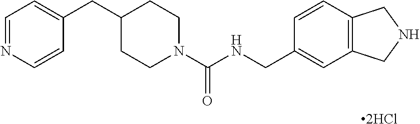 [SMILES: O=C(NCc1ccc2c(c1)CNC2)N1CCC(Cc2ccncc2)CC1 ; "·2HCl"] | N-(2,3-Dihydro-1H- isoindol-5- ylmethyl)-4-(pyridin-4- ylmethyl)piperidine-1- carboxamide dihydrochloride | 58 | 26% | Calculated for C21H26N4O: 350.210661, found 350.210901. HPLC: Rf 3.11 min, 100%. |
| 66 | 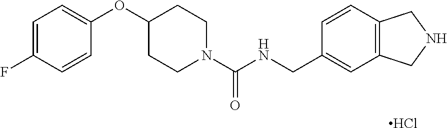 [SMILES: O=C(NCc1ccc2c(c1)CNC2)N1CCC(Oc2ccc(F)cc2)CC1 ; Cl] | N-(2,3-Dihydro-1H- isoindol-5- ylmethyl)-4-(4- fluorophenoxy) piperidine-1- carboxamide hydrochloride | 59 | 76% | Calculated for C₂₁H₂₄FN₃O₂: 369.185255, found 369.186415. HPLC: Rf 7.23 min, 99.4%. |
| 67 | 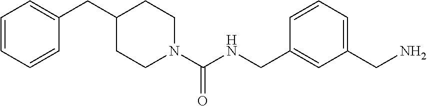 [SMILES: NCc1cccc(CNC(=O)N2CCC(Cc3ccccc3)CC2)c1] | N-{[3-(Aminometh- yl)phen- yl]methyl}- 4-benzylpiperidine- 1-carboxamide | 51 | 46% | Calculated for C₂₁H₂₇N₃O: 337.2154, found 337.2147. HPLC: Rf 5.07 min, 100%. |
| 68 | 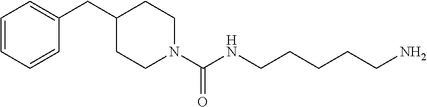 [SMILES: NCCCCCNC(=O)N1CCC(Cc2ccccc2)CC1] | N-(5-Aminopentyl)-4- benzylpiperidine-1- carboxamide | 54 | 24% | LCMS: purity 98%, ES⁺ 304.8 [MH]⁺. HPLC: Rf 4.85 min, 100%. |

### Example 69

### 2,2,2-Trifluoroacetic acid; N-({6-amino-1H-pyrrolo[3,2-c]pyridin-2-yl}methyl)-4-benzylpiperidine-1-carboxamide

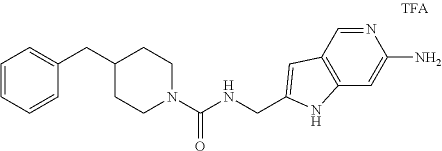 [SMILES: Nc1cc2[nH]c(CNC(=O)N3CCC(Cc4ccccc4)CC3)cc2cn1]

Intermediate 72 (40.0 mg, 0.08 mmol) was dissolved in DCM (1 mL) and TFA (1 mL) and stirred for 3 d. The solvents were removed in vacuo and the residue purified by reverse phase chromatography to give the title compound (1.13 mg, 3%) as a white solid. LCMS purity 100%, ES⁺ 364.0 [MH]⁺. HPLC: Rf 5.13 min, 90%.

### Example 70

### 2,2,2-Trifluoroacetic acid; 4-benzyl-N-{[4-(N′,N,N-trimethylcarbamimidoyl)phenyl]methyl}piperidine-1-carboxamide

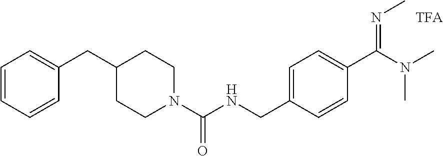 [SMILES: C/N=C(/c1ccc(CNC(=O)N2CCC(Cc3ccccc3)CC2)cc1)N(C)C]

Intermediate 76 (0.20 g, 0.55 mmol), POCl₃ (0.08 mL, 0.83 mmol) and DIPEA (0.11 mL, 0.66 mmol) were suspended in DCE (5 mL) and heated at reflux for 2 h. Dimethylamine hydrochloride (0.22 g, 2.75 mmol) was added and the mixture stirred at reflux for 5 days. The solvents were removed in vacuo and the product suspended in DCM (10 mL), sonicated and the solids removed by filtration. The filtrate was concentrated in vacuo and purified by HPLC (1% TFA) to afford the title compound (10.0 mg, 3.59%) as a colourless film. LCMS purity 100%, ES⁺ 393.0 [MH]⁺. HPLC: Rf 5.24 min, 97%.

### Example 71

### 2,2,2-Trifluoroacetic acid; 4-benzyl-N-({4-[(1Z)-(methylimino)(pyrrolidin-1-yl)methyl]phenyl}methyl)piperidine-1-carboxamide

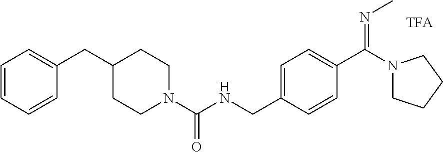 [SMILES: C/N=C(/c1ccc(CNC(=O)N2CCC(Cc3ccccc3)CC2)cc1)N1CCCC1]

Intermediate 75 (0.08 g, 0.22 mmol), POCl₃ (0.03 mL, 0.26 mmol) and DIPEA (0.04 mL, 0.26 mmol) were suspended in DCE (1 mL) and heated at reflux for 2 h. Pyrrolidine (0.09 mL, 1.10 mmol) was added and the mixture stirred at reflux overnight. The solvents were removed in vacuo and the product suspended in DCM (5 mL), sonicated, and the solids removed by filtration and the residue purified by chromatography. The product was dissolved in DCM (2 mL) and TFA (0.5 mL), stirred at room temperature for 1 h and concentrated in vacuo to afford the title compound (5.84 mg, 6.52%) as a colourless glass. LCMS purity 100%, ES⁺ 419.0 [MH]⁺. HPLC: Rf 5.45 min, 98%.

### Biological Tests

### PAR2 Studies

The PAR2 receptor couples through the Gq signaling pathway and results in activation of calcium mobilization. The functional activity of test compounds was routinely tested by measuring the ability of compounds to antagonize PAR2 (trypsin challenge) activity in a dose dependent manner, in 1321N1 cells transfected with the human PAR2 receptor, using a calcium flux Fluorescent Imaging Plate Reader FLIPR assay. To provide confirmation of functional inhibition, compounds were also examined at the native PAR2 receptor expressed in the A549 cell line.

The selectivity of compounds for PAR2 versus the PAR1 and PAR4 receptors was evaluated using the native 1321N1 cell line. In order to confirm that activity at the PAR2 receptor was due to direct inhibition of the PAR2 receptor as opposed to inhibition of trypsin, a series of serine protease assays was developed to measure the activity of in-house compounds on enzyme activity.

### Functional Calcium Mobilisation Studies

Briefly, test compounds were dissolved in DMSO to a concentration of 20 mM and stored in matrix screenmate racks. The required amount of compound was transferred to 96-well compound plates on the day of assay and diluted in assay buffer to the required final concentration; dose-response measurements were assayed by making 1:3.16 serial dilutions to produce 10 point curves. The compounds were then transferred to 384-well assay plates ready for use. Top concentrations were adjusted depending on the potency of the compounds with a typical concentration range of 200 μM to 6.3 nM being used. The assay buffer used was HBSS buffer supplemented with 20 mM HEPES and 0.1% BSA (protease free), pH7.4. The loading/wash buffers were the same as the assay buffer.

Human PAR2 transfected 1321N1 cells were cultured in Dulbecco's modified Eagles medium (DMEM) supplemented with 10% dialyzed FBS, 1% Penicillin/Streptomycin, 378.5 μg/ml Geneticin G418 sulphate and maintained at 37° C. in a humidified, 5% CO₂ controlled atmosphere. Sub-cultivations were performed every 2-3 d. At confluence the cells were lifted using Ca²⁺ and Mg²⁺ free PBS/0.02% (w/v) EDTA, spun at 1000 rpm for 3 min and re-suspended in medium at 2×10⁵ cells/mL, transferred (50 μl/well) to 384-well black/clear Costar plates (Costar #3712) and incubated at 37° C. in a 5% CO₂/95% air humidified incubator for 4 h. The cells were washed with assay buffer at 37° C. using the Biotek ELx 405, washing 3 times, leaving 20 μl buffer in the well. After washing, the cells were loaded with Fluo-4 AM dye (Molecular probes) at 2 μM containing 0.48 μg/mL pluronic acid for 60 min at 37° C. under 5% CO₂. Following the incubation, cells were washed in assay buffer at 37° C. using the Biotek ELx 405, washing 3 times, leaving 40 μl in each well and incubated for 10 min at 37° C. before use.

A combined agonist/antagonist protocol was used to measure changes in intracellular calcium concentration. Compound (antagonist) was added to the cell plate using a Fluorometric Imaging Plate Reader (FLIPR) (Molecular Devices, Sunnyvale, Calif., USA). Basal fluorescence was recorded every second for 10 seconds prior to compound addition (10 μl) and fluorescence recorded every second for 1 min then every 6 seconds for a further 1 min. Trypsin (EC₅₀ concentration) was then added using the FLIPR and fluorescence recorded as described above. Curve-fitting and parameter estimation were carried out using GraphPad Prism 4.0 (GraphPad Software Inc., San Diego, Calif.).

### Trypsin Enzyme Inhibition

The commercially available protease assay kit from Calbiochem (Cat #539125) was used to determine inhibition of trypsin activity. The kit quantifies trypsin activity by measuring the cleaved product of FTC-casein. To measure enzyme inhibition activity, compounds were pre-incubated with trypsin before the addition of substrate. Compound IC50 was determined as percentage inhibition of trypsin.

All of the exemplified compounds of the invention were found to be potent and selective inhibitors of PAR2 (See Table 7).

TABLE 7
PAR2 antagonist activity

| | PAR2 |
|---|---|
| Example | IC₅₀ |
| 1 | B |
| 2 | C |
| 3 | A |
| 4 | A |
| 5 | A |
| 6 | A |
| 7 | B |
| 8 | B |
| 9 | C |
| 10 | B |
| 11 | C |
| 12 | B |
| 13 | C |
| 14 | C |
| 15 | C |
| 16 | B |
| 17 | A |
| 18 | B |
| 19 | B |
| 20 | C |
| 21 | D |
| 22 | C |
| 23 | B |
| 24 | B |
| 25 | B |
| 26 | C |
| 27 | C |
| 28 | C |
| 29 | D |
| 30 | D |
| 31 | D |
| 32 | D |
| 33 | C |
| 34 | A |
| 35 | A |
| 36 | A |
| 37 | C |
| 38 | B |
| 39 | C |
| 40 | A |
| 41 | B |
| 42 | A |
| 43 | B |
| 44 | B |
| 45 | A |
| 47 | A |
| 50 | A |
| 51 | C |
| 52 | B |
| 53 | C |
| 54 | D |
| 55 | C |
| 56 | C |
| 57 | C |
| 58 | C |
| 59 | B |
| 60 | B |
| 61 | D |
| 62 | B |
| 63 | B |
| 64 | B |
| 65 | D |
| 66 | C |
| 67 | D |
| 68 | C |

(A: <5 · M, B: 5-20 · M, C: 20-50 · M, D: 50-100 · M)

1. A compound of formula (I) or a pharmaceutically acceptable salt, solvate, or hydrate thereof 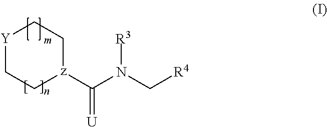
Y is —N(R^{1A})— or —C(R^{1B})(R²)—; and
R^{1A} is —X—R⁵ and R^{1B} is -Q-R⁵;
X is independently selected from a direct bond, —C(O)—, —(CHR⁶)ₚ—, —N(R⁶)— or, in either orientation, —(CH₂CHR⁶)—;
Q is independently selected from a direct bond, —O—, —S—, —N(R⁶)—, —C(O)—, C(H)(OH)—, —(CHR⁶)ₚ— or, in either orientation, —(CH₂CHR⁶)—;
p is 1 or 2;
U═O or S
R⁵ is a monocyclic aromatic or non-aromatic carbocyclic or heterocyclic ring having 5 or 6 ring atoms, optionally fused to a second aromatic or non-aromatic monocyclic carbocyclic or heterocyclic ring to form a 5-5, 5-6, 6-5, or 6-6 bicyclic ring system, which monocyclic ring or bicyclic ring system is optionally substituted with one more substituents independently selected from halogen, hydroxy, cyano, nitro, CF₃, C₁₋₄-alkyl, C₁₋₄-alkoxy and —NR^{7A}R^{7B}, wherein
R^{7A}, R^{7B} are each independently selected from hydrogen and C₁₋₄-alkyl, wherein any alkyl residue is optionally substituted with one or more substituents independently selected from fluorine, hydroxyl and C₁₋₄-alkoxy,
or
R^{7A} and R^{7B}, together with the nitrogen atom to which they are bound, form a 4- to 7-membered saturated heterocyclic ring, optionally substituted with one or more substituents independently selected from fluorine, hydroxyl, C₁₋₄-alkyl, fluoro-C₁₋₄-alkyl and C₁₋₄-alkoxy;
R² is H,
Z is N, and the ring comprising Z and Y is optionally substituted,
n=0, 1, or 2, and m=0 or 1, provided that m=0 when n=2, and provided that neither m nor n=0 when Z and Y are each N, and
R³ and R⁶ are each independently selected from H, C₁₋₄ alkyl, or cyclopropyl each of which C₁₋₄ alkyl, or cyclopropyl being optionally substituted with one or more substituents independently selected from fluoro, and C₁₋₄ alkoxy;
R⁴ is
(i) a 6-5 bicyclic ring system selected from 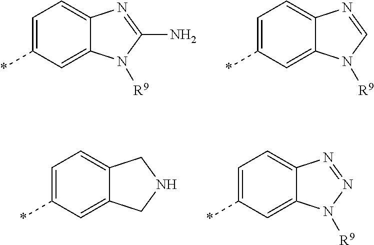
optionally substituted on either ring, and wherein the bond marked * is connected to the CH₂, or
(ii) the 5-6 bicyclic ring system 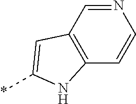
optionally substituted on either ring, and wherein the bond marked * is connected to the CH₂, or
(iii) a radical of formula —(W)ᵥ(CH₂)ₜB
wherein W is an optionally substituted phenyl or pyridyl ring, v is 0 or 1, and t is 0 or 3 provided that when v=0, t=3, and when v=1, t=0; and
B is selected from: 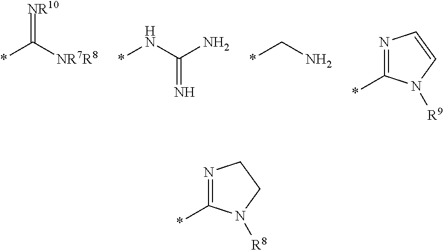
wherein R⁷, R⁸, R⁹ and R¹⁰ are independently selected from H, C₁₋₄ alkyl, or cyclopropyl, each of which C₁₋₄ alkyl, or cyclopropyl being optionally substituted with one or more substituents independently selected from fluoro and C₁₋₄ alkoxy; or
R⁷ and R⁸ together with the nitrogen atom to which they are attached form a 3-5 membered heterocyclic ring selected from aziridine, azetidine, and pyrrolidine each of which being optionally substituted with one or more substituents independently selected from fluoro and C₁₋₄ alkoxy.

2. A compound according to claim 1 wherein R⁷ and R⁸ are independently selected from H, C₁₋₄ alkyl, or cyclopropyl, each of which C₁₋₄ alkyl, or cyclopropyl being optionally substituted with one or more substituents independently selected from fluoro and C₁₋₄ alkoxy

3. A compound according to claim 1 wherein the ring comprising Z and Y is selected from: 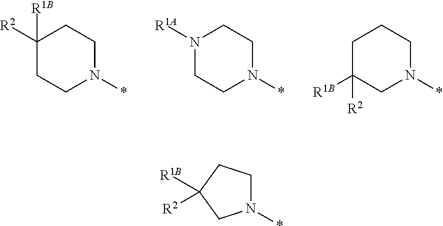
wherein the bond marked * connects to the carbon of the carbonyl group.

4. A compound according to claim 1 wherein the ring comprising Z and Y is optionally substituted with one more substituents independently selected from fluoro, C₁₋₄-alkyl, C₁₋₄-alkoxy, fluoro-C₁₋₄-alkyl and fluoro-C₁₋₄-alkoxy.

5. A compound according to claim 1 wherein the radical —(W)ᵥ(CH₂)ₜB is selected from: 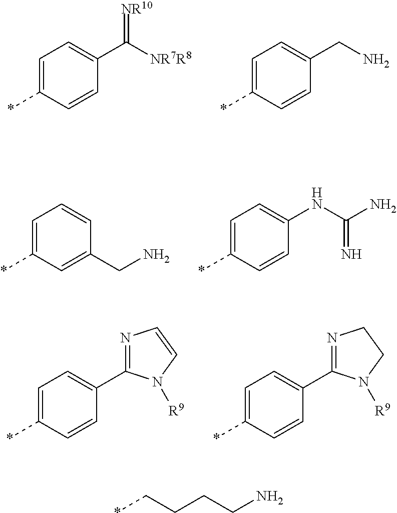
any of which being optionally substituted, and wherein the bond marked * is connected to the CH₂.

6. A compound according to claim 1 wherein R⁴ is selected from: 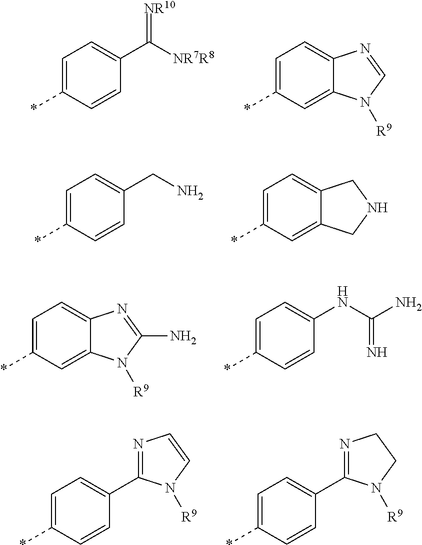
any of which being optionally substituted, and wherein the bond marked * is connected to the CH₂.

7. A compound according to any claim 1 wherein R¹⁰ is hydrogen.

8. A compound according to claim 1 wherein W is an optionally substituted phenyl ring.

9. A compound according to claim 1 wherein the R⁴ substituent is optionally substituted with one or more fluoro substituents

10. A compound according to claim 1 wherein R³ is H.

11. A compound according to claim 1 wherein R⁶ is H or methyl.

12. A compound according to claim 1 wherein R⁹ is H or methyl.

13. A compound 1 wherein R⁵ is selected from: 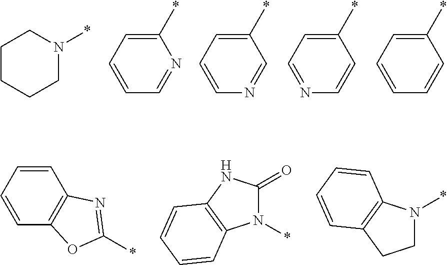
wherein the bond marked * connects R⁵ to the rest of the molecule, each of which being optionally substituted by the optional substituents defined in claim 1.

14. (canceled)

15. A compound according to claim 1 wherein U═O.

16. A compound according to claim 1 wherein X is independently selected from —C(O)—, —(CHR⁶)ₚ—, —N(R⁶)— or, in either orientation, —(CH₂CHR⁶)—.

17. A compound according to claim 1 wherein Q is independently selected from —O—, —S—, —N(R⁶)—, —C(O)—, C(H)(OH)—, —(CHR⁶)ₚ— or, in either orientation, —(CH₂CHR⁶)—.

18. A compound according to claim 1 wherein X is independently selected from —C(O)—, —(CHR⁶)ₚ—, or —N(R⁶).

19. A compound according to claim 1 wherein Q is independently selected from —O—, —N(R⁶)—, —C(O)—, C(H)OH)—, —(CHR⁶)ₚ—.

20. A compound as claimed in claim 1 selected from:
(4-{[(4-Benzylpiperidin-1-yl)carbonylamino]methyl}phenyl)methanaminium chloride;
2,2,2-Trifluoroacetic acid; N-{[4-(aminomethyl)phenyl]methyl}-4-[(4-methoxyphenyl)methyl]piperidine-1-carboxamide:
2,2,2-Trifluoroacetic acid; N-{[4-(aminomethyl)phenyl]methyl}-4-[(3-fluorophenyl)methyl]piperidine-1-carboxamide;
N-{[4-(Aminomethyl)phenyl]methyl}-4-[(4-fluorophenyl)methyl]piperidine-1-carboxamide hydrochloride;
2,2,2-Trifluoroacetic acid; N-{[4-(aminomethyl)phenyl]methyl}-4-[(4-chlorophenyl)methyl]piperidine-1-carboxamide;
2,2,2-Trifluoroacetic acid; N-{[4-(aminomethyl)phenyl]methyl}-4-[(4-methylphenyl)methyl]piperidine-1-carboxamide
N-{[4-(Aminomethyl)phenyl]methyl}-4-(pyridin-2-ylmethyl)piperidine-1-carboxamide;
N-{[4-(Aminomethyl)phenyl]methyl}-4-(pyridin-4-ylmethyl)piperidine-1-carboxamide;
N-{[4-(Aminomethyl)phenyl]methyl}-4-(4-fluorophenoxy)piperidine-1-carboxamide;
N-{[4-(Aminomethyl)phenyl]methyl}-4-(phenylsulfanyl)piperidine-1-carboxamide hydrochloride;
N-{[4-(Aminomethyl)phenyl]methyl}-4-[(2-chlorophenyl)amino]piperidine-1-carboxamide dihydrochloride;
2,2,2-Trifluoroacetic acid; N-{[4-(aminomethyl)phenyl]methyl}-4-[(4-fluorophenyl)carbonyl]piperidine-1-carboxamide;
N-{[4-(Aminomethyl)phenyl]methyl}-4-[(4-fluorophenyl)(hydroxy)methyl]piperidine-1-carboxamide;
N-{[4-(Aminomethyl)phenyl]methyl}-1-[(3-fluorophenyl)methyl]piperidine-4-carboxamide;
N-{[4-(Aminomethyl)phenyl]methyl}-4-benzylpiperazine-1-carboxamide;
N-{[b 4-(Aminomethyl)phenyl]methyl}-4-[(2-chlorophenyl)methyl]piperazine-1-carboxamide dihydrochloride;
N-{[4-(Aminomethyl)phenyl]methyl}-4-(1,3-benzoxazol-2-yl)piperidine-1-carboxamide;
N-{[4-(Aminomethyl)-3-fluorophenyl]methyl}-4-benzylpiperidine-1-carboxamide;
4-Benzyl-N-[(4-carbamimidoylphenyl)methyl]piperidine-1-carboxamide;
2,2,2-Trifluoroacetic acid; 4-benzyl-N-{[4-(N,N-dimethylcarbamimidoyl)phenyl]methyl}piperidine-1-carboxamide:
N-[(4-Carbamimidoylphenyl)methyl]-4-(pyridin-4-ylmethyl)piperidine-1-carboxamide;
N-(1H-1,3-Benzodiazol-6-ylmethyl)-4-benzylpiperidine-1-carboxamide;
2,2,2-Trifluoroacetic acid; N-(1H-1,3-benzodiazol-5-ylmethyl)-3-phenylpyrrolidine-1-carboxamide;
2,2,2-Trifluoroacetic acid; N-(1H-1,3-b-benzodiazol-5-ylmethyl)-3-benzylpyrrolidine-1-carboxamide;
N-[(2-Amino-1H-1,3-benzodiazol-6-yl)methyl]-4-benzylpiperidine-1-carboxamide;
2,2,2-Tri fluoroacetic acid; 4-benzyl-N-[(4-carbamimidamidophenyl)methyl]piperidine-1-carboxamide;
4-Benzyl-N-(2,3-dihydro-1H-isoindol-5-ylmethyl)piperidine-1-carboxamide

21. A pharmaceutical composition comprising a compound as claimed in claim 1, together with a pharmaceutically acceptable carrier.

22. The use of a compound of formula (I) as claimed in claim 1 in the preparation of a composition for the treatment of diseases or conditions responsive to the reduction of PAR2 mediated activity.

23. The use as claimed in claim 22 for the reduction of PAR2 mediated activity, ex vivo or in vivo.

24. The use as claimed in claim 22 wherein the diseases or conditions are selected from inflammation, intestinal inflammation, inflammatory skin diseases including psoriasis and itch, fibrosis, arthritis, pain, cancer and pancreatitis.

25. A method for the treatment of diseases or conditions responsive to the reduction of PAR2 mediated activity, which comprises administering to a subject suffering such disease an effective amount of a compound of formula (I) as claimed in claim 1.

26. A method as claimed in claim 25 for the treatment of inflammation, intestinal inflammation, inflammatory skin diseases including psoriasis and itch, fibrosis, arthritis, pain, cancer and pancreatitis.
